SEQUENCE LISTING (20 sequences)

SEQ ID NO: 1 - protein, 75 aa, Homo sapiens
CPPPMSVEHADIWVKSYSLYSRERYICNSGFKRKAGTSSLTECVLNKATNVAHWTTPSLKCIRDPALVHQRPAPP

SEQ ID NO: 2 - protein, 23 aa, Artificial
  Synthetic Linker
DTTEPATPTTPVTTPTTTDDLDA

SEQ ID NO: 3 - protein, 116 aa, Homo sapiens
LDNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 214 aa, Artificial
  Synthetic IL-15Ra_Flex_IL-15 in HC sequence
CPPPMSVEHADIWVKSYSLYSRERYICNSGFKRKAGTSSLTECVLNKATNVAHWTTPSLKCIRDPALVHQRPAPPDTTEPATPTTPVTTPTTTDDLDALDNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 27 aa, Artificial
  Synthetic Linker
QTPTNTISVTPTNNSTPTNNSNPKPNP

SEQ ID NO: 6 - protein, 33 aa, Artificial
  Synthetic Linker
ASQTPTNTISVTPTNNSTPTNNSNPKPNPDIGM

SEQ ID NO: 7 - protein, 120 aa, Artificial [sequence omitted]

SEQ ID NO: 8 - protein, 113 aa, Artificial [sequence omitted]

SEQ ID NO: 9 - protein, 117 aa, Artificial
  Synthetic VH domain of the anti-PD1Gepi 135c
QVQLVQSGAEVKKPGASVKMSCKASGYTFTNFYIHWVRQAPGQGLEWIGSIYPNYGDTAYNQKFKDRATLTVDTSTSTAYMELSSLRSEDTAVYYCARGYSYAMDYWGQGTLVTVSS

SEQ ID NO: 10 - protein, 440 aa, Artificial
  Synthetic heavy chain of pembrolizumab
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 447 aa, Artificial
  Synthetic heavy chain of nivolumab
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 444 aa, Artificial
  Synthetic heavy chain of the anti-PD1Gepi 135c
QVQLVQSGAEVKKPGASVKMSCKASGYTFTNFYIHWVRQAPGQGLEWIGSIYPNYGDTAYNQKFKDRATLTVDTSTSTAYMELSSLRSEDTAVYYCARGYSYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 694 aa, Artificial
  Synthetic C3711 or C3721 [hKeytruda(anti-PD-1)-HC-LV-hIgG4H-C-Flex-v1-hIL-15Ra-hIL-15]
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASQTPTNTISVTPTNNSTPTNNSNPKPNPDIGMCPPPMSVEHADIWVKSYSLYSRERYICNSGFKRKAGTSSLTECVLNKATNVAHWTTPSLKCIRDPALVHQRPAPPDTTEPATPTTPVTTPTTTDDLDALDNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 14 - protein, 694 aa, Artificial
  Synthetic [nivolumab (anti-PD-1)-HC-LV-hIgG4H- C-Flex-v1-hIL-15Ra-hIL-15]
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASQTPTNTISVTPTNNSTPTNNSNPKPNPDIGMCPPPMSVEHADIWVKSYSLYSRERYICNSGFKRKAGTSSLTECVLNKATNVAHWTTPSLKCIRDPALVHQRPAPPDTTEPATPTTPVTTPTTTDDLDALDNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 15 - protein, 691 aa, Artificial
  Synthetic C3789 [hHC anti?PD1Gepi 135c-LV- hIgG4H-C-Flex-v1-hIL-15Ra-hIL-15]
QVQLVQSGAEVKKPGASVKMSCKASGYTFTNFYIHWVRQAPGQGLEWIGSIYPNYGDTAYNQKFKDRATLTVDTSTSTAYMELSSLRSEDTAVYYCARGYSYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASQTPTNTISVTPTNNSTPTNNSNPKPNPDIGMCPPPMSVEHADIWVKSYSLYSRERYICNSGFKRKAGTSSLTECVLNKATNVAHWTTPSLKCIRDPALVHQRPAPPDTTEPATPTTPVTTPTTTDDLDALDNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 16 - protein, 218 aa, Artificial
  Synthetic light chain of pembrolizumab
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 214 aa, Artificial
  Synthetic light chain of nivolumab
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 214 aa, Artificial
  Synthetic light chain of the anti-PD1Gepi 135c antibody
DIQMTQSPSSLSASVGDRVTITCSASQGISGDLNWYQQKPGKAVKLLIYHTSSLHSGVPSRFSGSGSGTDYTLTISSLQPEDFATYYCQYYSKDLLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 2157 bp, Artificial
  Synthetic nucleic acid encoding for C3711 or C3721 [hKeytruda(anti-PD-1)-HC-LV-hIgG4H-C-Flex-v1-hIL-15Ra-hIL- 15]
atggatccgaaaggttctctgagctggcgtattctgctgttcctaagcctggcgttcgaactgtcttatggtcaggttcagctggttcagtctggagttgaagtgaaaaaaccgggcgcgtctgttaaagtttcttgcaaagcgtctggttacaccttcaccaactactacatgtactgggttcgtcaggcgccgggtcagggcctggaatggatgggcggtatcaacccgtctaacggtggcaccaacttcaacgaaaaattcaaaaaccgtgttaccctgaccaccgatagcagcaccaccaccgcgtatatggaactgaaatctctgcagttcgacgacaccgcagtgtactactgcgcccgccgcgactaccgtttcgacatgggcttcgactactgggggcaaggtaccacagttaccgtatcgagcgccagcacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagccagacccccaccaacaccatcagcgtgacccccaccaacaacagcacccccaccaacaacagcaaccccaagcccaaccccgatatcgggatgtgccctccccccatgtccgtggaacacgcagacatctgggtcaagagctacagcttgtactccagggagcggtacatttgtaactctggtttcaagcgtaaagccggcacgtccagcctgacagagtgcgtgttgaacaaggccacgaatgtcgcccactggacaacccccagtctcaaatgcattagagaccctgccctggttcaccaaaggccagcgccacccgatacaacagaacctgcaacacctacaacacctgtaacaacaccgacaacaacagatgatctggatgcactcgacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagctccaggtaattagcttagaatctggcgacgccagcatccacgatacagttgagaatctgatcattctcgcaaacaactctctgtcttctaacggcaacgtgacagagtctggctgtaaggagtgtgaggagctggaggagaagaacatcaaggagtttctgcagtcttttgtgcacatcgtgcagatgtttatcaacacctcttga

SEQ ID NO: 20 - DNA, 2133 bp, Artificial
  Synthetic nucleic acid encoding for C3789 [hHC anti?PD1Gepi 135c-LV-hIgG4H-C-Flex-v1-hIL-15Ra-hIL-15]
atgggatggtcttggattctgctgtttttgttgagcgtgacagctggagtgcatagccaggtgcagttggtgcagagcggagccgaggtgaagaaacctggagcctccgtgaagatgagctgtaaggctagtggatacacttttacaaacttttacattcattgggtgaggcaggcccccggccaggggctggagtggatcggcagcatctaccccaactacggcgataccgcctacaaccagaagttcaaggatagggccaccctgacagtggacaccagcactagcacagcctacatggagctgagcagcctgcggagcgaggacacagcggtgtactactgcgccaggggctacagctacgctatggattactgggggcaggggaccctggtgacagtgagcagcgccagcacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagccagacccccaccaacaccatcagcgtgacccccaccaacaacagcacccccaccaacaacagcaaccccaagcccaaccccgatatcgggatgtgccctccccccatgtccgtggaacacgcagacatctgggtcaagagctacagcttgtactccagggagcggtacatttgtaactctggtttcaagcgtaaagccggcacgtccagcctgacagagtgcgtgttgaacaaggccacgaatgtcgcccactggacaacccccagtctcaaatgcattagagaccctgccctggttcaccaaaggccagcgccacccgatacaacagaacctgcaacacctacaacacctgtaacaacaccgacaacaacagatgatctggatgcactcgacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagctccaggtaattagcttagaatctggcgacgccagcatccacgatacagttgagaatctgatcattctcgcaaacaactctctgtcttctaacggcaacgtgacagagtctggctgtaaggagtgtgaggagctggaggagaagaacatcaaggagtttctgcagtcttttgtgcacatcgtgcagatgtttatcaacacctcttga

(19) United States
(12) Patent Application Publication
(10) Pub. No.: US 2023/0034677 A1
(43) Pub. Date: Feb. 2, 2023

(54) NOVEL IMMUNOTHERAPIES TARGETING PD-1 WITH ANTI-PD-1/IL-15 IMMUNOCYTOKINES

(71) Applicants: INSERM (Institut National de la Santé et la Recherche (Médicale), PARIS (FR); Assistance Publique-Hôpitaux de Paris (APHP), Paris (FR); Université Paris-Est Créteil val de Marne, Creteil (FR); Baylor Research Institutue, Dallas, TX (US); Mabquest, Pully (CH)

(72) Inventors: Yves LEVY, Creteil (FR); Giuseppe PANTALEO, Pully (CH); Craig FENWICK, Pully (CH); Sandra ZURAWSKI, Dallas, TX (US); Gérard ZURAWSKI, Dallas, TX (US); Nabila SEDDIKI, Creteil (FR)

(21) Appl. No.: 17/778,617

(22) PCT Filed: Nov. 20, 2020
(86) PCT No.: PCT/EP2020/082892

§ 371 Date: May 20, 2022

(30) Foreign Application Priority Data
Nov. 21, 2019 (EP) 19306499.5

(51) Int. Cl. C07K 14/54; C07K 14/715; A61P 35/00

(52) U.S. Cl. CPC C07K 14/5443; C07K 14/7155; A61P 35/00; C07K 2319/00; C07K 2317/52

(57) ABSTRACT

The inventors now provide novel IL-15/IL-15 receptor alpha (IL-15Rα) fusion proteins. Furthermore, as a complement to anti-PD-1 therapy, the inventors developed a series of anti-PD-1/IL-15/IL-15 receptor alpha (IL-15Rα) immunocytokines that are able to simultaneously target multiple steps in the immune activation process. The development of said immunocytokines provides the potential benefits associated with anti-PD-1 antibodies and IL-15 administered individually with several distinct advantages. These include a significantly extended in vivo half-life relative to the IL-15 therapy, administration of a pre-formed IL-15/IL-15Rα complex that would preclude the need for IL-15Rα trans-presentation, high activity leading to a low target therapeutic dose and targeted delivery of IL-15 to regions with high PD-1 cells that will limit off-target adverse events.

### FIELD OF THE INVENTION

The present invention is in the field of medicine, in particular, immunology, oncology and virology.

### BACKGROUND OF THE INVENTION

Antibodies targeting PD-1 are the gold standard immunotherapy strategy for oncology and hundreds of clinical trials are ongoing to identify combination therapies that are more effective and/or more broadly applicable to different forms of cancer. Therapies can contribute to an anti-tumor effect at three specific immune enhancing steps. These are generally to: 1) promote sufficient number and diversity of tumor primed T cells, 2) ensure proper trafficking to and penetration of the tumor and, 3) prevent the inhibition of the tumor specific T cell response. Combinations of therapies that target different limiting steps in the immune mediated anti-tumor response are predicted to have a synergetic therapeutic effect. This hypothesis was confirmed in trials that combined Opdivo anti-PD-1 therapy to relieve functional exhaustion of tumor-specific CD8⁺ T cells with Ipilimumab that acts through CTLA-4 blockade to enhance T cell priming through antigen presenting cells. Although therapeutic benefits were observed for several forms of cancer, patients also face a considerable increase in adverse events that has limited the use of this immunotherapy combination. As such, immunotherapy combinations have significant potential in enhancing the anti-tumor immune response but the safety of these combinations is an important consideration.

Therapeutic vaccines and immunomodulators have been commonly used to enhance HIV-1-specific cell-mediated immune responses and to suppress virus replication. While the former is important to stimulate HIV-1-specific T-cell responses, the latter may support the expansion of the stimulated virus-specific T cells and render them more cytotoxic for virus clearance. The recent success of the immunotherapies targeting PD-1 in the cancer field provide the scientific basis for developing potent combinatorial therapeutics to allow enhanced immune response to gain more efficient control of virus replication.

WO/2007/046006 discloses IL-15/IL-15 receptor alpha (IL-15Rα) fusion proteins that can promote tumor clearance by multiple distinct ways compared to the anti-PD-1 therapy. These include the activation and induced proliferation of NK cells, induced proliferation of antigen specific CD8⁺ T cells, enhanced cytolytic activity of CD8⁺ T cells and induction of long-lasting antigen-experienced CD8⁺CD44hi memory T cells. An additional benefit is that in contrast to IL-2, IL-15 doesn't induce the proliferation of Tregs that exert a suppressive effect on the expansion of tumor specific CTL. Support for the use of anti-PD-1 Abs in combination with said IL-15/IL-15 receptor alpha (IL-15Rα) fusion proteins has been shown using a mouse in vivo tumor model where animals treated with the combination therapy had a significant reduction in the rate of tumor growth and a prolonged survival relative to either monotherapy administered alone (Desbois, Mélanie, et al. “IL-15 trans-signaling with the superagonist RLI promotes effector/memory CD8⁺T cell responses and enhances antitumor activity of PD-1 antagonists.” The Journal of Immunology 197.1 (2016): 168-178.). The mechanism associated with this synergy is in part due to the immune enhancement of T cell signaling pathways that promote antigen-specific stimulation, cell proliferation and cell survival. Despite these benefits, the use of IL-15/IL-15 receptor alpha (IL-15Rα) fusion proteins has only limited use in the clinic due to several limitations. These include: 1) a short in vivo half-life (<1 hrs; Stoklasek et al. 2006) leading to high Cmax values, 2) a low dose limiting toxicity due to adverse events including vascular leak syndrome, 3) the requirement for the IL-15Rα trans-presentation which is often downregulated in the tumor micro-environment, 4) the rapid upregulation of PD-1 on T cells that leads to a self-limiting immune response when used as a monotherapy and 5) severe but reversible neutropenia due to the high therapeutic doses used.

WO20012/175222 discloses an immunocytokine comprising (a) a conjugate, and (b) an antibody or a fragment thereof directly or indirectly linked by covalence to said conjugate, wherein said conjugate comprises (i) a polypeptide comprising the amino acid sequence of the interleukin 15 or derivatives thereof, and a polypeptide comprising the amino acid sequence of the sushi domain of the IL-15Rα or derivatives thereof. However, the prior art does not disclose anti-PD-1/IL-15 immunocytokines.

### SUMMARY OF THE INVENTION

As defined by the claims, the present invention relates to novel IL-15/IL-15 receptor alpha (IL-15Rα) fusion proteins, immunocytokines that comprises said fusion proteins and uses of said immunocytokines for the treatment of cancer and viral infections.

### DETAILED DESCRIPTION OF THE INVENTION

The inventors now provide novel IL-15/IL-15 receptor alpha (IL-15Rα) fusion proteins. Furthermore, as a complement to anti-PD-1 therapy, the inventors developed a series of anti-PD-1/IL-15/IL-15 receptor alpha (IL-15Rα) immunocytokines that are able to simultaneously target multiple steps in the immune activation process. The development of said immunocytokines provides the potential benefits associated with anti-PD-1 antibodies and IL-15 administered individually with several distinct advantages. These include a significantly extended in vivo half-life relative to the IL-15 therapy, administration of a pre-formed IL-15/IL-15Rα complex that would preclude the need for IL-15Rα trans-presentation, high activity leading to a low target therapeutic dose and targeted delivery of IL-15 to regions with high PD-1 cells that will limit off-target adverse events.

Most importantly, the IL-15/IL-15 receptor alpha (IL-15Rα) fusion proteins of the present invention differ from the fusion proteins described in WO007046006 by the nature of the linker. The linker sequence (“Flex sequence”) of the present invention does not respect the features put forward by the teaching of WO007046006. Indeed, WO007046006 teaches that the linker sequence shall have minimal hydrophobic or charged character in order to promote interaction with the functional protein domains, and preferably comprises glycine (G), asparagine (N) and serine (S). Here, the inventors surprisingly show that the linker of the present invention that is constituted mainly by hydrophobic (such as alanine A, valine V, proline P and leucine L) or charged amino acids (such as aspartic acid D and glutamic acid E) and that does not comprises glycine (G), asparagine (N) and serine (S) however leads to obtaining functional fusion proteins.

### Main Definitions

As used herein, the term “PD-1” has its general meaning in the art and refers to programmed cell death protein 1 (also known as CD279). PD-1 acts as an immune checkpoint, which upon binding of one of its ligands, PD-L1 or PD-L2, enables Shp2 to dephosphorylate CD28 and inhibits the activation of T cells.

As used herein, the term “CD40” has its general meaning in the art and refers to human CD40 polypeptide receptor. In some embodiments, CD40 is the isoform of the human canonical sequence as reported by UniProtKB-P25942 (also referred as human TNR5).

As used herein, the term “interleukin 15” has its general meaning in the art and refers to a cytokine with structural similarity to IL-2 (GRABSTEIN et al., Science, vol. 264(5161), p:965-968, 1994). This cytokine is also known as IL-15, IL15 or MGC9721. This cytokine and IL-2 share many biological activities and they were found to bind common hematopoietin receptor subunits. Thus, they may compete for the same receptor, negatively regulating each other's activity. It has been established that IL-15 regulates T and natural killer cells activation and proliferation, and that the number of CD8⁺ memory T cells is shown to be controlled by a balance between this cytokine and IL-2. IL-15 activity can be measured by determining its proliferation induction on kit225 cell line (HORI et al., Blood, vol. 70(4), p: 1069-72, 1987).

As used herein the term “the sushi domain of IL-15Rα” has its general meaning in the art and refers to a domain beginning at the first cysteine residue (C1) after the signal peptide of IL-15Rα, and ending at the fourth cysteine residue (C4) after said signal peptide. Said sushi domain corresponding to a portion of the extracellular region of IL-15Rα is necessary for its binding to IL-15 (WEI et al., J Immunol., vol. 167(1), p: 277-282, 2001).

As used herein, the terms “peptide”, “polypeptide”, and “protein” are used interchangeably, and refer to a compound comprised of amino acid residues covalently linked by peptide bonds. A polypeptide is not limited to a specific length: it must contain at least two amino acids, and no limitation is placed on the maximum number of amino acids that can comprise a polypeptide's sequence. Peptides, oligopeptides, and proteins are included within the definition of polypeptide, and such terms may be used interchangeably herein unless specifically indicated otherwise. As used herein, the term refers to both short chains, which also commonly are referred to in the art as peptides, oligopeptides and oligomers, for example, and to longer chains, which generally are referred to in the art as proteins, of which there are many types. In one embodiment, as used herein, the term “peptides” refers to a linear polymer of amino acids linked together by peptide bonds, preferably having a chain length of less than about 50 amino acids residues; a “polypeptide” refers to a linear polymer of at least 50 amino acids linked together by peptide bonds; and a protein specifically refers to a functional entity formed of one or more peptides or polypeptides, optionally glycosylated, and optionally of non-polypeptides cofactors. This term also does exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof. Particular polypeptides of interest in the context of this invention are amino acid subsequences comprising CDRs and being capable of binding an antigen.

As used herein, the term “fusion protein” refers to a recombinant protein comprising at least one polypeptide chain which is obtained or obtainable by genetic fusion, for example by genetic fusion of at least two gene fragments encoding separate functional domains of distinct proteins. Thus, the term “fusion protein” refers to a construct wherein two polypeptides are fused directly or via a linker. As used herein, the term “directly” means that the (first or last) amino acid at the terminal end (N or C-terminal end) of the first polypeptide is fused to the (first or last) amino acid at the terminal end (N or C-terminal end) of the second polypeptide. In other words, in this embodiment, the last amino acid of the C-terminal end of said polypeptide is directly linked by a covalent bond to the first amino acid of the N-terminal end of said heterologous polypeptide, or the first amino acid of the N-terminal end of said polypeptide is directly linked by a covalent bond to the last amino acid of the C-terminal end of said heterologous polypeptide.

As used herein, the term “linker” has its general meaning in the art and refers to an amino acid sequence of a length sufficient to ensure that the proteins form proper secondary and tertiary structures. In some embodiments, the linker is a peptidic linker which comprises at least one, but less than 30 amino acids e.g., a peptidic linker of 2-30 amino acids, preferably of 10-30 amino acids, more preferably of 15-30 amino acids, still more preferably of 19-27 amino acids, most preferably of 20-26 amino acids. In some embodiments, the linker has 2; 3; 4; 5; 6; 7; 8; 9; 10; 11; 12; 13; 14; 15; 16; 17; 18; 19; 20; 21; 22; 23; 24; 25; 26; 27; 28; 29; 30 amino acid residues. Typically, linkers are those which allow the compound to adopt a proper conformation (i.e., a conformation allowing a proper signal transducing activity through the IL-15Rbeta/gamma signalling pathway). Usually, the most suitable linker sequences (1) will adopt a flexible extended conformation, (2) will not exhibit a propensity for developing ordered secondary structure which could interact with the functional domains of fusion proteins, and (3) will have minimal hydrophobic or charged character which could promote interaction with the functional protein domains.

As used herein, the term “nucleic acid” or “polynucleotide” refers to a polymer of nucleotides covalently linked by phosphodiester bonds, such as deoxyribonucleic acids (DNA) or ribonucleic acids (RNA), in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Typically, the nucleic acids of the present invention are codon optimized. As used herein, the term, “optimized” means that a nucleotide sequence has been altered to encode an amino acid sequence using codons that are preferred in the production cell or organism, generally a eukaryotic cell, for example, a Chinese Hamster Ovary cell (CHO) or a human cell. The optimized nucleotide sequence is engineered to retain completely or as much as possible the amino acid sequence originally encoded by the starting nucleotide sequence. The amino acid sequences encoded by optimized nucleotide sequences are also referred to as optimized.

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase “nucleotide sequence that encodes a protein or a RNA” may also include introns to the extent that the nucleotide sequence encoding the protein may in some version contain an intron(s).

As used herein, the terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence.

As used herein, the term “promoter/regulatory sequence” refers to a nucleic acid sequence (such as, for example, a DNA sequence) recognized by the synthetic machinery of the cell, or introduced synthetic machinery, required to initiate the specific transcription of a polynucleotide sequence, thereby allowing the expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

As used herein, the term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

As used herein, the term “expression system” means a host cell and compatible vector under suitable conditions, e.g., for the expression of a protein coded for by foreign DNA carried by the vector and introduced to the host cell.

As used herein, the “percent identity” between the two sequences is a function of the number of identical positions shared by the sequences (i. e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (NEEDLEMAN, and Wunsch). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification.

According to the invention a first amino acid sequence having at least 80% of identity with a second amino acid sequence means that the first sequence has 80; 81; 82; 83; 84; 85; 86; 87; 88; 89; 90; 91; 92; 93; 94; 95; 96; 97; 98; 99 or 100% of identity with the second amino acid sequence.

As used herein, the term “antibody” has its general meaning in the art and refers to immunoglobulin molecules and immunologically active portions of immunoglobulin molecules, i.e., molecules that contain an antigen binding site that immunospecifically binds to an antigen. In natural antibodies of rodents and primates, two heavy chains are linked to each other by disulfide bonds, and each heavy chain is linked to a light chain by a disulfide bond. There are two types of light chains, lambda (l) and kappa (k). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate in the antibody binding site, or influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. For the agonist antibodies described hereafter, the CDRs have been determined using CDR finding algorithms from www.bioinf.org.uk—see the section entitled «How to identify the CDRs by looking at a sequence» within the Antibodies pages.

As used herein, the term “IgG Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody. Accordingly, a composition of antibodies of the invention may comprise antibody populations with all K447 residues removed, antibody populations with no K447 residues removed, and antibody populations having a mixture of antibodies with and without the K447 residue.

As used herein, the terms “monoclonal antibody”, “monoclonal Ab”, “monoclonal antibody composition”, “mAb”, or the like, as used herein refer to a preparation of antibody molecules of single molecular composition. A monoclonal antibody is obtained from a population of substantially homogeneous antibodies, i.e., the individual antibodies comprised in the population are identical except for possible naturally occurring mutations that may be present in minor amounts. A monoclonal antibody composition displays a single binding specificity and affinity for a particular epitope. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with the appropriate antigenic forms (i.e., PD-1 polypeptides). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. However, the modifier “monoclonal” is not to be construed as requiring production of the antibody by any particular method. For example, a monoclonal antibody may also be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). A “monoclonal antibody” may also be isolated from phage antibody libraries using the techniques described in Clackson et al., Nature, 352:624-628 (1991) and Marks et al., J. Mol. Biol., 222:581-597 (1991), for example.

As used herein, the term “chimeric antibody” refers to an antibody which comprises a VH domain and a VL domain of a non-human antibody, and a CH domain and a CL domain of a human antibody. In one embodiment, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used herein, the term “humanized antibody” include antibodies which have the 6 CDRs of a murine antibody, but humanized framework and constant regions. More specifically, the term “humanized antibody”, as used herein, may include antibodies in which CDR sequences derived from the germline of another mammalian species, such as a mouse, have been grafted onto human framework sequences.

As used herein the term “human monoclonal antibody”, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, in one embodiment, the term “human monoclonal antibody”, as used herein, is not intended to include antibodies in which CDR sequences derived from the germline of another mammalian species, such as a mouse, have been grafted onto human framework sequences.

As used herein, the term “K_{assoc}” or “Kₐ” is intended to refer to the association rate of a particular antibody-antigen interaction, whereas the term “K_{dis}” or “K_{d},” as used herein, is intended to refer to the dissociation rate of a particular antibody-antigen interaction.

As used herein, the term “K_{D}” is intended to refer to the dissociation constant, which is obtained from the ratio of K_{d} to Kₐ (i.e., K_{d}/Kₐ) and is expressed as a molar concentration (M). Methods for measuring the K_{D} of an antibody are well known in the art and include, without limitation, surface plasmon resonance (SPR) technology in a BIAcore 3000 instrument using a soluble form of the antigen as the ligand and the antibody as the analyte. BIACORE® (GE Healthcare, Piscaataway, N.J.) is one of a variety of surface plasmon resonance assay formats that are routinely used to epitope bin panels of monoclonal antibodies. Affinities of antibodies can be readily determined using other conventional techniques, for example, those described by Scatchard et al., (Ann. N.Y. Acad. Sci. USA 51:660 (1949)). Binding properties of an antibody to antigens, cells or tissues may generally be determined and assessed using immunodetection methods including, for example, immunofluorescence-based assays, such as immunohistochemistry (IHC) and/or fluorescence-activated cell sorting (FACS). Typically, an antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein), which is not identical or closely related to the predetermined antigen. When the K_{D} of the antibody is very low (that is, the antibody has a high affinity), then the K_{D} with which it binds the antigen is typically at least 10,000-fold lower than its K_{D} for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

As used herein, the term “binding specificity” refers to the ability of an antibody to detectably bind to an antigen recombinant polypeptide, such as recombinant PD-1 polypeptide, with a K_{D} of 100 nM or less, 10 nM or less, 5 nM or less, as measured by Surface Plasmon Resonance (SPR) measurements.

As used herein, the term “specificity” refers to the ability of an antibody to detectably bind an epitope presented on an antigen, such as a PD-1 or CD40, while having relatively little detectable reactivity with non-PD-1 or CD40 proteins. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a PD-1 or CD40). The term “affinity”, as used herein, means the strength of the binding of an antibody to an epitope. The affinity of an antibody is given by the dissociation constant Kd, defined as [Ab]×[antigen]/[Ab-antigen], where [Ab-antigen] is the molar concentration of the antibody-antigen complex, [Ab] is the molar concentration of the unbound antibody and [antigen] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

As used herein, the term “epitope” refers to a specific arrangement of amino acids located on a protein or proteins to which an antibody binds. Epitopes often consist of a chemically active surface grouping of molecules such as amino acids or sugar side chains, and have specific three dimensional structural characteristics as well as specific charge characteristics. Epitopes can be linear or conformational, i.e., involving two or more sequences of amino acids in various regions of the antigen that may not necessarily be contiguous.

As used herein, the term “immunocytokine” refers to an antibody directly or indirectly linked by covalence to a cytokine or derivates thereof. Said antibody and said cytokine can be linked by a linker.

As used herein, the term” T cells” has its general meaning in the art and represent an important component of the immune system that plays a central role in cell-mediated immunity. T cells are known as conventional lymphocytes as they recognize the antigen with their TCR (T cell receptor for the antigen) with presentation or restriction by molecules of the complex major histocompatibility. There are several subsets of T cells each having a distinct function such as CD8⁺ T cells, CD4⁺ T cells, Gamma delta T cells, and Tregs.

As used herein, the term “CD8⁺ T cell” has its general meaning in the art and refers to a subset of T cells which express CD8 on their surface. They are WIC class I-restricted, and function as cytotoxic T cells. “CD8⁺ T cells” are also called cytotoxic T lymphocytes (CTL), T-killer cells, cytolytic T cells, or killer T cells. CD8 antigens are members of the immunoglobulin supergene family and are associative recognition elements in major histocompatibility complex class I-restricted interactions. As used herein, the term “tumor infiltrating CD8⁺ T cell” refers to the pool of CD8⁺ T cells of the patient that have left the blood stream and have migrated into a tumor.

As used herein, the term “CD4⁺ T cells” (also called T helper cells or TH cells) refers to T cells which express the CD4 glycoprotein on their surfaces and which assist other white blood cells in immunologic processes, including maturation of B cells into plasma cells and memory B cells, and activation of cytotoxic T cells and macrophages. CD4⁺ T cells become activated when they are presented with peptide antigens by WIC class II molecules, which are expressed on the surface of antigen-presenting cells (APCs). Once activated, they divide rapidly and secrete cytokines that regulate or assist in the active immune response. These cells can differentiate into one of several subtypes, including TH1, TH2, TH3, TH17, TH9, TFH or Treg, which secrete different cytokines to facilitate different types of immune responses. Signaling from the APC directs T cells into particular subtypes. In addition to CD4, the TH cell surface biomarkers known in the art include CXCR3 (Th1), CCR4, Crth2 (Th2), CCR6 (Th17), CXCRS (Tfh) and as well as subtype-specific expression of cytokines and transcription factors including T-bet, GATA3, EOMES, RORγT, BCL6 and FoxP3.

As used herein, the term “gamma delta T cell” has its general meaning in the art. Gamma delta T cells normally account for 1 to 5% of peripheral blood lymphocytes in a healthy individual (human, monkey). They are involved in mounting a protective immune response, and it has been shown that they recognize their antigenic ligands by a direct interaction with antigen, without any presentation by MEW molecules of antigen-presenting cells. Gamma 9 delta 2 T cells (sometimes also called gamma 2 delta 2 T cells) are gamma delta T cells bearing TCR receptors with the variable domains Vγ9 and Vδ2. They form the majority of gamma delta T cells in human blood. When activated, gamma delta T cells exert potent, non-MHC restricted cytotoxic activity, especially efficient at killing various types of cells, particularly pathogenic cells. These may be cells infected by a virus (Poccia et al., J. Leukocyte Biology, 1997, 62: 1-5) or by other intracellular parasites, such as mycobacteria (Constant et al., Infection and Immunity, December 1995, vol. 63, no. 12: 4628-4633) or protozoa (Behr et al., Infection and Immunity, 1996, vol. 64, no. 8: 2892-2896). They may also be cancer cells (Poccia et al., J. Immunol., 159: 6009-6015; Fournie and Bonneville, Res. Immunol., 66th Forum in Immunology, 147: 338-347). The possibility of modulating the activity of said cells in vitro, ex vivo or in vivo would therefore provide novel, effective therapeutic approaches in the treatment of various pathologies such as infectious diseases (particularly viral or parasitic), cancers, allergies, and even autoimmune and/or inflammatory disorders.

As used herein the term “CAR-T cell” refers to a T lymphocyte that has been genetically engineered to express a CAR. The definition of CAR T-cells encompasses all classes and subclasses of T-lymphocytes including CD4⁺, CD8⁺ T cells, gamma delta T cells as well as effector T cells, memory T cells, regulatory T cells, and the like. The T lymphocytes that are genetically modified may be “derived” or “obtained” from the subject who will receive the treatment using the genetically modified T cells or they may “derived” or “obtained” from a different subject.

As used herein, the term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an antigen binding domain to an intracellular signaling domain. In some embodiments, the stimulatory molecule is the zeta chain associated with the T cell receptor complex. In some embodiments, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule. In some embodiments, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In some embodiments, the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. In particular aspects, CARs comprise fusions of single-chain variable fragments (scFv) derived from monoclonal antibodies, fused to CD3-zeta a transmembrane domain and endodomain. In some embodiments, CARs comprise domains for additional co-stimulatory signaling, such as CD3-zeta, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

As used herein, the term “T cell exhaustion” refers to a state of T cell dysfunction. The T cell exhaustion generally arises during many chronic infections and cancer. T cell exhaustion can be defined by poor effector function, sustained expression of inhibitory receptors, and/or a transcriptional state distinct from that of functional effector or memory T cells. T cell exhaustion generally prevents optimal control of infection and tumors. See, e.g., Wherry E J, Nat Immunol. (2011) 12: 492-499, for additional information about T cell exhaustion. Typically, T cell exhaustion results from the binding of PD-1 to its ligands PD-L1 and PD-L2.

As used herein, “Dendritic Cells” or “DCs” refers to any member of a diverse population of morphologically similar cell types found in lymphoid or non-lymphoid tissues. These cells are characterized by their distinctive morphology and high levels of surface MHC-class II expression. These cells can be isolated from a number of tissue sources, and conveniently, from peripheral blood, as described in the Examples below.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the present invention contemplate any one or more of these aspects of treatment. In one embodiment, the terms “treating” or “treatment” refers to both therapeutic treatment and prophylactic or preventative measures; wherein the object is to prevent or slow down (lessen) the targeted disease. Therefore, in one embodiment, those in need of treatment may include those already with the disorder as well as those prone to have the disorder or those in whom the disorder is to be prevented.

As used herein, the term “cancer” has its general meaning in the art and includes, but is not limited to, solid tumors and blood borne tumors The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxo sarcoma; liposarcoma; leiomyo sarcoma; rhabdomyo sarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; NK leukemia or NK lymphoma, such as for example, extranodal and non-extranodal NK/T lymphomas; NK cell derived malignancies; and acute NK leukemia; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

As used herein the term “infectious disease” includes any infection caused by viruses, bacteria, protozoa, molds or fungi. In some embodiments, the viral infection comprises infection by one or more viruses selected from the group consisting of Arenaviridae, Astroviridae, Birnaviridae, Bromoviridae, Bunyaviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, Tymoviridae, Hepadnaviridae, Herpesviridae, Paramyxoviridae or Papillomaviridae viruses. Relevant taxonomic families of RNA viruses include, without limitation, Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, and Tymoviridae viruses. In some embodiments, the viral infection comprises infection by one or more viruses selected from the group consisting of adenovirus, rhinovirus, hepatitis, immunodeficiency virus, polio, measles, Ebola, Coxsackie, Rhino, West Nile, small pox, encephalitis, yellow fever, Dengue fever, influenza (including human, avian, and swine), lassa, lymphocytic choriomeningitis, junin, machuppo, guanarito, hantavirus, Rift Valley Fever, La. Crosse, California encephalitis, Crimean-Congo, Marburg, Japanese Encephalitis, Kyasanur Forest, Venezuelan equine encephalitis, Eastern equine encephalitis, Western equine encephalitis, severe acute respiratory syndrome (SARS), parainfluenza, respiratory syncytial, Punta Toro, Tacaribe, pachindae viruses, adenovirus, Dengue fever, influenza A and influenza B (including human, avian, and swine), junin, measles, parainfluenza, Pichinde, punta toro, respiratory syncytial, rhinovirus, Rift Valley Fever, severe acute respiratory syndrome (SARS), Tacaribe, Venezuelan equine encephalitis, West Nile and yellow fever viruses, tick-borne encephalitis virus, Japanese encephalitis virus, St. Louis encephalitis virus, Murray Valley virus, Powassan virus, Rocio virus, louping-ill virus, Banzi virus, Ilheus virus, Kokobera virus, Kunjin virus, Alfuy virus, bovine diarrhea virus, and Kyasanur forest disease. Bacterial infections that can be treated according to this invention include, but are not limited to, infections caused by the following: Staphylococcus; Streptococcus, including S. pyogenes; Enterococcl; Bacillus, including Bacillus anthracis, and Lactobacillus; Listeria; Corynebacterium diphtherias; Gardnerella including G. vaginalis; Nocardia; Streptomyces; Thermoactinomyces vulgaris; Treponerna; Camplyobacter, Pseudomonas including aeruginosa; Legionella; Neisseria including N. gonorrhoeae and N. meningitides; Flavobacterium including F. meningosepticum and F. odoraturn; Brucella; Bordetella including B. pertussis and B. bronchiseptica; Escherichia including E. coli, Klebsiella; Enterobacter, Serratia including S. marcescens and S. liquefaciens; Edwardsiella; Proteus including P. mirabilis and P. vulgaris; Streptobacillus; Rickettsiaceae including R. fickettsfi, Chlamydia including C. psittaci and C. trachornatis; Mycobacterium including M. tuberculosis, M. intracellulare, M. folluiturn, M. laprae, M. avium, M. bovis, M. africanum, M. kansasii, M. intracellulare, and M. lepraernurium; and Nocardia. Protozoa infections that may be treated according to this invention include, but are not limited to, infections caused by leishmania, kokzidioa, and trypanosoma. A complete list of infectious diseases can be found on the website of the National Center for Infectious Disease (NCID) at the Center for Disease Control (CDC) (World Wide Web (www) at cdc.gov/ncidod/diseases/), which list is incorporated herein by reference. All of said diseases are candidates for treatment using the compositions according to the invention.

As used herein, the term “antigen” or “Ag” as used herein refers to protein, peptide, nucleic acid or tissue or cell preparations capable of eliciting a T cell response.

As used herein, the term “therapeutically effective amount” refers to an amount effective, at dosages and for periods of time necessary, to achieve a desired therapeutic result. Thus, the terms “therapeutically effective amount” may mean a level or amount of antibodies that is aimed at, without causing significant negative or adverse side effects to the target, (1) delaying or preventing the onset of the targeted disease; (2) slowing down or stopping the progression, aggravation, or deterioration of one or more symptoms of the targeted disease; (3) bringing about ameliorations of the symptoms of the targeted disease; (4) reducing the severity or incidence of the targeted disease; or (5) curing the targeted disease. A therapeutically effective amount may be administered prior to the onset of the targeted disease, for a prophylactic or preventive action. Alternatively, or additionally, the therapeutically effective amount may be administered after initiation of the targeted disease, for a therapeutic action.

As used herein, the term “pharmaceutically acceptable carrier” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, for example, FDA Office or EMA.

As used herein, the term “vaccine” is intended to mean a composition which can be administered to humans or to animals in order to induce an immune response; this immune response can result in a production of antibodies or simply in the activation of certain cells, in particular antigen-presenting cells, T lymphocytes and B lymphocytes. In some embodiments the vaccine is capable of producing an immune response that leads to the production of neutralizing antibodies in the patient with respect to the antigen provided in the vaccine. The vaccine can be a composition for prophylactic purposes or for therapeutic purposes, or both.

As used herein, the term “subject” refers to a warm-blooded animal, preferably a mammal (including humans, domestic and farm animals, and zoo, sports, or pet animals, such as dogs, cats, cattle, horses, sheep, pigs, goats, rabbits, etc. . . . ), and more preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. In one embodiment, the subject is an adult (for example a subject above the age of 18). In another embodiment, the subject is a child (for example a subject below the age of 18). In one embodiment, the subject is a male. In another embodiment, the subject is a female.

Novel IL-15/IL-15 Receptor Alpha (IL-15Rα) Fusion Proteins of the Present Invention:

The first object of the present invention relates to an IL-15/IL-15 receptor alpha (IL-15Rα) fusion protein comprising i) a IL-15-Ralpha sushi-containing polypeptide comprising an amino acid sequence having at least 80% of identity with the amino acid sequence of SEQ ID NO:1 ii) a linker having an amino acid sequence as set forth in SEQ ID NO:2 and iii) an IL-15 polypeptide comprising the amino acid sequence having at least at least 80% of identity with the amino acid sequence of SEQ ID NO:3.

>IL-15Ra sushi sequence
SEQ ID NO: 1
CPPPMSVEHADIWVKSYSLYSRERYICNSGFKRKAGTSSLTECVLNKATN
VAHWTTPSLKCIRDPALVHQRPAPP
>Flex sequence
SEQ ID NO: 2
DTTEPATPTTPVTTPTTTDDLDA
>IL-15 sequence
SEQ ID NO: 3
LDNWVNVISDLKKIEDLIQSMHIDATLYTESDVHPSCKVTAMKCFLLELQ
VISLESGDASIHDTVENLIILANNSLSSNGNVTESGCKECEELEEKNIKE
FLQSFVHIVQMFINTS

According to the present invention, the IL-15-Ralpha sushi-containing polypeptide, the linker and the IL-15 polypeptide are fused in frame wherein the C-terminal end of the IL15-Ralpha sushi-containing polypeptide is fused to the N-terminal end of the linker and the C-terminal end of the linker is fused to the N-terminal end of the IL-15 polypeptide.

In some embodiments, the IL-15/IL-15 receptor alpha (IL-15Rα) fusion protein of the present invention that consists of an amino acid sequence as set forth in SEQ ID NO:4.

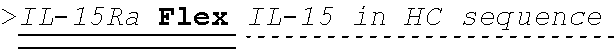

SEQ ID NO: 4
CPPPMSVEHADIWVKSYSLYSRERYICNSGFKRKAGTSSLTECVL
NKATNVAHWTTPSLKCIRDPALVH

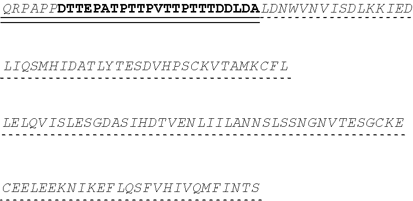

According to the invention, the IL-15/IL-15 receptor alpha fusion protein of the present invention is produced by conventional automated peptide synthesis methods or by recombinant expression. General principles for designing and making proteins are well known to those of skill in the art. The IL-15/IL-15 receptor alpha fusion protein of the present invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. The IL-15/IL-15 receptor alpha (IL-15Rα) fusion protein of the present invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art. As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides. A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In some embodiments, it is contemplated that the IL-15/IL-15 receptor alpha fusion protein of the present invention is modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. For example, Pegylation is a well-established and validated approach for the modification of a range of polypeptides. The benefits include among others: (a) markedly improved circulating half-lives in vivo due to either evasion of renal clearance as a result of the polymer increasing the apparent size of the molecule to above the glomerular filtration limit, and/or through evasion of cellular clearance mechanisms; (b) reduced antigenicity and immunogenicity of the molecule to which PEG is attached; (c) improved pharmacokinetics; (d) enhanced proteolytic resistance of the conjugated protein; and (e) improved thermal and mechanical stability of the PEGylated polypeptide.

Immunocytokines of the Present Invention:

A further object of the present invention relates to a heavy chain of an antibody that is fused to the IL-15/IL-15 receptor alpha fusion protein of the present invention.

In some embodiments, the heavy chain is fused to the IL-15/IL-15 receptor alpha fusion protein via a linker. In some embodiments, the linker comprises the amino acid sequence as set forth in SEQ ID NO:5 (i.e., FlexV1 linker). In some embodiments, the linker consists of the amino acid sequence as set forth in SEQ ID NO:6.

>FlexV1 linker
SEQ ID NO: 5
QTPTNTISVTPTNNSTPTNNSNPKPNP
>linker
SEQ ID NO: 6
ASQTPTNTISVTPTNNSTPTNNSNPKPNPDIGM

In some embodiments, the heavy chain comes from an antibody having specificity of PD-1. In some embodiments, the anti-PD-1 antibody is an already known antibody or is a novel antibody.

To select novel anti-PD-1 antibodies, a variety of methods of screening antibodies have been described in the Art. Such methods may be divided into in vivo systems, such as transgenic mice capable of producing fully human antibodies upon antigen immunization and in vitro systems, consisting of generating antibody DNA coding libraries, expressing the DNA library in an appropriate system for antibody production, selecting the clone that express antibody candidate that binds to the target with the affinity selection criteria and recovering the corresponding coding sequence of the selected clone. These in vitro technologies are known as display technologies, and include without limitation, phage display, RNA or DNA display, ribosome display, yeast or mammalian cell display. They have been well described in the Art (for a review see for example: Nelson et al., 2010 Nature Reviews Drug discovery, “Development trends for human monoclonal antibody therapeutics” (Advance Online Publication) and Hoogenboom et al. in Method in Molecular Biology 178:1-37, O'Brien et al., ed., Human Press, Totowa, N.J., 2001). In one some embodiment, human recombinant Anti-PD-1 antibodies are isolated using phage display methods for screening libraries of human recombinant antibody libraries with PD-1 binding. Repertoires of VH and VL genes or related CDR regions can be separately cloned by polymerase chain reaction (PCR) or synthesized by DNA synthesizer and recombined randomly in phage libraries, which can then be screened for antigen-binding clones. Such phage display methods for isolating human antibodies are established in the art or described in the examples below. See for example: U.S. Pat. Nos. 5,223,409; 5,403,484; and U.S. Pat. No. 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

In some embodiments, human antibodies directed against PD-1 can be identified using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.” The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al., 1994 Nature 368(6474): 856-859). In another embodiment, human anti-PD-1 antibodies can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

In some embodiments, the antibody is a chimeric antibody, in particular a chimeric mouse/human antibody.

In some embodiments, the antibody is humanized antibody.

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, ω®, and SCH-900475), and CT-011 (also known as Pidilizumab, hBAT, and hBAT-1). In some embodiments, the PD-1 binding antagonist is AMP-224 (also known as B7-DCIg).

In some embodiments, the anti-PD-1 antibody is the anti-PD1Gepi 135c as disclosed in WO2016020856 and in Fenwick, Craig, et al. “Tumor suppression of novel anti-PD-1 antibodies mediated through CD28 costimulatory pathway.” Journal of Experimental Medicine (2019): jem-20182359.

In some embodiments, the heavy chain of the present invention comprises the VH domain as set forth in SEQ ID NO:7, 8 or 9.

>VH domain of pembrolizumab
SEQ ID NO: 7
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSS
>VH domain of nivolumab
SEQ ID NO: 8
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSS
>VH domain of the anti-PD1Gepi 135c
SEQ ID NO: 9
QVQLVQSGAEVKKPGASVKMSCKASGYTFTNFYIHWVRQAPGQGLEWIGS
IYPNYGDTAYNQKFKDRATLTVDTSTSTAYMELSSLRSEDTAVYYCARGY
SYAMDYWGQGTLVTVSS

In some embodiments, the heavy chain comprises an IgG Fc region of an IgG4 immunoglobulin.

In some embodiments, the heavy chain consists of the amino acid sequence as set forth in SEQ ID NO:10, 11 or 12.

>heavy chain of pembrolizumab
SEQ ID NO: 10
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK
>heavy chain of nivolumab
SEQ ID NO: 11
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
<heavy chain of the anti-PD1Gepi 135c
SEQ ID NO: 12
QVQLVQSGAEVKKPGASVKMSCKASGYTFTNFYIHWVRQAPGQGLEWIGS
IYPNYGDTAYNQKFKDRATLTVDTSTSTAYMELSSLRSEDTAVYYCARGY
SYAMDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFEGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK

In some embodiments, the heavy chain of an antibody that is fused to the IL-15/IL-15 receptor alpha fusion protein of the present invention consists of the amino acid sequence as set forth in SEQ ID NO:13, 14, or 15.

>C3711 or C3721 [hKeytruda(anti-PD-1)-HC-LV-
hIgG4H-C-Flex-v1-hIL-15Ra-hIL-15]
SEQ ID NO: 13
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFEGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKASQ
TPTNTISVTPTNNSTPTNNSNPKPNPDIGMCPPPMSVEHADIWVKSYSLY
SRERYICNSGFKRKAGTSSLTECVLNKATNVAHWTTPSLKCIRDPALVHQ
RPAPPDTTEPATPTTPVTTPTTTDDLDALDNWVNVISDLKKIEDLIQSMH
IDATLYTESDVHPSCKVTAMKCFLLELQVISLESGDASIHDTVENLIILA
NNSLSSNGNVTESGCKECEELEEKNIKEFLQSFVHIVQMFINTS
>[nivolumab (anti-PD-1)-HC-LV-hIgG4H-C-Flex-v1-
hIL-15Ra-hIL-15]
SEQ ID NO: 14:
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKASQ
TPTNTISVTPTNNSTPTNNSNPKPNPDIGMCPPPMSVEHADIWVKSYSLY
SRERYICNSGFKRKAGTSSLTECVLNKATNVAHWTTPSLKCIRDPALVHQ
RPAPPDTTEPATPTTPVTTPTTTDDLDALDNWVNVISDLKKIEDLIQSMH
IDATLYTESDVHPSCKVTAMKCFLLELQVISLESGDASIHDTVENLIILA
NNSLSSNGNVTESGCKECEELEEKNIKEFLQSFVHIVQMFINTS
>C3789 [hHC anti-PD1Gepi 135c-LV-hIgG4H-C-Flex-
v1-hIL-15Ra-hIL-15]
SEQ ID NO: 15
QVQLVQSGAEVKKPGASVKMSCKASGYTFTNFYIHWVRQAPGQGLEWIGS
IYPNYGDTAYNQKFKDRATLTVDTSTSTAYMELSSLRSEDTAVYYCARGY
SYAMDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFEGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGKASQTPT
NTISVTPTNNSTPTNNSNPKPNPDIGMCPPPMSVEHADIWVKSYSLYSRE
RYICNSGFKRKAGTSSLTECVLNKATNVAHWTTPSLKCIRDPALVHQRPA
PPDTTEPATPTTPVTTPTTTDDLDALDNWVNVISDLKKIEDLIQSMHIDA
TLYTESDVHPSCKVTAMKCFLLELQVISLESGDASIHDTVENLIILANNS
LSSNGNVTESGCKECEELEEKNIKEFLQSFVHIVQMFINTS

A further object of the present invention relates to an immunocytokine that comprises a heavy chain of an antibody that is fused to the IL-15/IL-15 receptor alpha fusion protein of the present invention.

In some embodiments, the immunocytokine of the present invention has specificity for PD-1. Thus in some embodiments, the immunocytokine of the present invention comprises a heavy chain as described above.

In some embodiments, the immunocytokine of the present invention comprises a heavy chain that consists of the amino acid sequence as set forth in SEQ ID NO:13, 14, or 15.

In some embodiments, the immunocytokine of the present invention comprises a heavy chain a set forth in SEQ ID NO:13 and a light chain a set forth in SEQ ID NO:16.

In some embodiments, the immunocytokine of the present invention comprises a heavy chain a set forth in SEQ ID NO:14 and a light chain a set forth in SEQ ID NO:17.

In some embodiments, the immunocytokine of the present invention comprises a heavy chain a set forth in SEQ ID NO:15 and a light chain a set forth in SEQ ID NO:18.

>light chain of pembrolizumab
SEQ ID NO: 16
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
>light chain of nivolumab
SEQ ID NO: 17
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
light chain of the the anti-PDIGepi 135c antibody
SEQ ID NO: 18:
DIQMTQSPSSLSASVGDRVTITCSASQGISGDLNWYQQKPGKAVKLLIYH
TSSLHSGVPSRFSGSGSGTDYTLTISSLQPEDFATYYCQYYSKDLLTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC

In some embodiments, the immunocytokine of the present invention that comprises a heavy chain a set forth in SEQ ID NO:15 and a light chain a set forth in SEQ ID NO:18 recognizes a PD-1 epitope that is located on the opposing face of the PDL-1 interaction. Thus according to this embodiment, the immunocytokine does not block the binding of PD-1 to its ligand (PD-L1 or PD-L2). Conversely, the immunocytokine can target PD-1 on cells that are already pre-bound with either pembrolizumab or nivolumab blocking anti-PD-1 antibodies. As such, the immunocytokine can be used in combination therapy either of these commercial antibodies without competing for binding to PD-1. The immunocytokine also has the potential to bind PD-1 on cells that are pre-complexed with either the PD-L1 or the PD-L2 ligand that may be expressed on either an antigen-presenting cell or a tumor cell. The anti-PD-1 portion of the immunocytokine acts primarily through the CD28 co-stimulatory receptor restoring signaling to the AKT pathway and calcium mobilization during T cell stimulation.

Nucleic Acids, Vectors and Host Cells of the Present Invention:

A further object of the invention relates to a nucleic acid that encodes for the IL-15/IL-15 receptor alpha (IL-15Rα) fusion protein of the present invention.

A further object of the present invention relates to a nucleic acid that encodes for a heavy chain of an antibody that is fused to the IL-15/IL-15 receptor alpha (IL-15Rα) fusion protein of the present invention.

In some embodiments, the nucleic acid comprises the nucleic acid sequence as set forth in SEQ ID NO:19 or 20.

>nucleic acid encoding for C3711 or C3721
[hKeytruda(anti-PD-1)-HC-LV-hIgG4H-C-Flex-v1-
hIL-15Ra-hIL-15]
SEQ ID NO: 19
ATGGATCCGAAAGGTTCTCTGAGCTGGCGTATTCTGCTGTTCCTAAGCCT
GGCGTTCGAACTGTCTTATGGTCAGGTTCAGCTGGTTCAGTCTGGAGTTG
AAGTGAAAAAACCGGGCGCGTCTGTTAAAGTTTCTTGCAAAGCGTCTGGT
TACACCTTCACCAACTACTACATGTACTGGGTTCGTCAGGCGCCGGGTCA
GGGCCTGGAATGGATGGGCGGTATCAACCCGTCTAACGGTGGCACCAACT
TCAACGAAAAATTCAAAAACCGTGTTACCCTGACCACCGATAGCAGCACC
ACCACCGCGTATATGGAACTGAAATCTCTGCAGTTCGACGACACCGCAGT
GTACTACTGCGCCCGCCGCGACTACCGTTTCGACATGGGCTTCGACTACT
GGGGGCAAGGTACCACAGTTACCGTATCGAGCGCCAGCACGAAGGGCCCA
TCCGTCTTCCCCCTGGCGCCCTGCTCCAGGAGCACCTCCGAGAGCACAGC
CGCCCTGGGCTGCCTGGTCAAGGACTACTTCCCCGAACCGGTGACGGTGT
CGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTC
CAGCAGCTTGGGCACGAAGACCTACACCTGCAACGTAGATCACAAGCCCA
GCAACACCAAGGTGGACAAGAGAGTTGAGTCCAAATATGGTCCCCCATGC
CCACCCTGCCCAGCACCTGAGTTCGAAGGGGGACCATCAGTCTTCCTGTT
CCCCCCAAAACCCAAGGACACTCTCATGATCTCCCGGACCCCTGAGGTCA
CGTGCGTGGTGGTGGACGTGAGCCAGGAAGACCCCGAGGTCCAGTTCAAC
TGGTACGTGGATGGCGTGGAGGTGCATAATGCCAAGACAAAGCCGCGGGA
GGAGCAGTTCAACAGCACGTACCGTGTGGTCAGCGTCCTCACCGTCCTGC
ACCAGGACTGGCTGAACGGCAAGGAGTACAAGTGCAAGGTCTCCAACAAA
GGCCTCCCGTCCTCCATCGAGAAAACCATCTCCAAAGCCAAAGGGCAGCC
CCGAGAGCCACAGGTGTACACCCTGCCCCCATCCCAGGAGGAGATGACCA
AGAACCAGGTCAGCCTGACCTGCCTGGTCAAAGGCTTCTACCCCAGCGAC
ATCGCCGTGGAGTGGGAGAGCAATGGGCAGCCGGAGAACAACTACAAGAC
CACGCCTCCCGTGCTGGACTCCGACGGCTCCTTCTTCCTCTACAGCAGGC
TAACCGTGGACAAGAGCAGGTGGCAGGAGGGGAATGTCTTCTCATGCTCC
GTGATGCATGAGGCTCTGCACAACCACTACACACAGAAGAGCCTCTCCCT
GTCTCTGGGTAAAGCTAGCCAGACCCCCACCAACACCATCAGCGTGACCC
CCACCAACAACAGCACCCCCACCAACAACAGCAACCCCAAGCCCAACCCC
GATATCGGGATGTGCCCTCCCCCCATGTCCGTGGAACACGCAGACATCTG
GGTCAAGAGCTACAGCTTGTACTCCAGGGAGCGGTACATTTGTAACTCTG
GTTTCAAGCGTAAAGCCGGCACGTCCAGCCTGACAGAGTGCGTGTTGAAC
AAGGCCACGAATGTCGCCCACTGGACAACCCCCAGTCTCAAATGCATTAG
AGACCCTGCCCTGGTTCACCAAAGGCCAGCGCCACCCGATACAACAGAAC
CTGCAACACCTACAACACCTGTAACAACACCGACAACAACAGATGATCTG
GATGCACTCGACAACTGGGTGAATGTAATAAGTGATTTGAAAAAAATTGA
AGATCTTATTCAATCTATGCATATTGATGCTACTTTATATACGGAAAGTG
ATGTTCACCCCAGTTGCAAAGTAACAGCAATGAAGTGCTTTCTCTTGGAG
CTCCAGGTAATTAGCTTAGAATCTGGCGACGCCAGCATCCACGATACAGT
TGAGAATCTGATCATTCTCGCAAACAACTCTCTGTCTTCTAACGGCAACG
TGACAGAGTCTGGCTGTAAGGAGTGTGAGGAGCTGGAGGAGAAGAACATC
AAGGAGTTTCTGCAGTCTTTTGTGCACATCGTGCAGATGTTTATCAACAC
CTCTTGA
>nucleic acid encoding for C3789 [hHC anti-
PD1Gepi 135c-LV-hIgG4H-C-Flex-v1-hIL-15Ra-hIL-15]
SEQ ID NO: 20
ATGGGATGGTCTTGGATTCTGCTGTTTTTGTTGAGCGTGACAGCTGGAGT
GCATAGCCAGGTGCAGTTGGTGCAGAGCGGAGCCGAGGTGAAGAAACCTG
GAGCCTCCGTGAAGATGAGCTGTAAGGCTAGTGGATACACTTTTACAAAC
TTTTACATTCATTGGGTGAGGCAGGCCCCCGGCCAGGGGCTGGAGTGGAT
CGGCAGCATCTACCCCAACTACGGCGATACCGCCTACAACCAGAAGTTCA
AGGATAGGGCCACCCTGACAGTGGACACCAGCACTAGCACAGCCTACATG
GAGCTGAGCAGCCTGCGGAGCGAGGACACAGCGGTGTACTACTGCGCCAG
GGGCTACAGCTACGCTATGGATTACTGGGGGCAGGGGACCCTGGTGACAG
TGAGCAGCGCCAGCACGAAGGGCCCATCCGTCTTCCCCCTGGCGCCCTGC
TCCAGGAGCACCTCCGAGAGCACAGCCGCCCTGGGCTGCCTGGTCAAGGA
CTACTTCCCCGAACCGGTGACGGTGTCGTGGAACTCAGGCGCCCTGACCA
GCGGCGTGCACACCTTCCCGGCTGTCCTACAGTCCTCAGGACTCTACTCC
CTCAGCAGCGTGGTGACCGTGCCCTCCAGCAGCTTGGGCACGAAGACCTA
CACCTGCAACGTAGATCACAAGCCCAGCAACACCAAGGTGGACAAGAGAG
TTGAGTCCAAATATGGTCCCCCATGCCCACCCTGCCCAGCACCTGAGTTC
GAAGGGGGACCATCAGTCTTCCTGTTCCCCCCAAAACCCAAGGACACTCT
CATGATCTCCCGGACCCCTGAGGTCACGTGCGTGGTGGTGGACGTGAGCC
AGGAAGACCCCGAGGTCCAGTTCAACTGGTACGTGGATGGCGTGGAGGTG
CATAATGCCAAGACAAAGCCGCGGGAGGAGCAGTTCAACAGCACGTACCG
TGTGGTCAGCGTCCTCACCGTCCTGCACCAGGACTGGCTGAACGGCAAGG
AGTACAAGTGCAAGGTCTCCAACAAAGGCCTCCCGTCCTCCATCGAGAAA
ACCATCTCCAAAGCCAAAGGGCAGCCCCGAGAGCCACAGGTGTACACCCT
GCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAAT
GGGCAGCCGGAGAACAACTACAAGACCACGCCTCCCGTGCTGGACTCCGA
CGGCTCCTTCTTCCTCTACAGCAGGCTAACCGTGGACAAGAGCAGGTGGC
AGGAGGGGAATGTCTTCTCATGCTCCGTGATGCATGAGGCTCTGCACAAC
CACTACACACAGAAGAGCCTCTCCCTGTCTCTGGGTAAAGCTAGCCAGAC
CCCCACCAACACCATCAGCGTGACCCCCACCAACAACAGCACCCCCACCA
ACAACAGCAACCCCAAGCCCAACCCCGATATCGGGATGTGCCCTCCCCCC
ATGTCCGTGGAACACGCAGACATCTGGGTCAAGAGCTACAGCTTGTACTC
CAGGGAGCGGTACATTTGTAACTCTGGTTTCAAGCGTAAAGCCGGCACGT
CCAGCCTGACAGAGTGCGTGTTGAACAAGGCCACGAATGTCGCCCACTGG
ACAACCCCCAGTCTCAAATGCATTAGAGACCCTGCCCTGGTTCACCAAAG
GCCAGCGCCACCCGATACAACAGAACCTGCAACACCTACAACACCTGTAA
CAACACCGACAACAACAGATGATCTGGATGCACTCGACAACTGGGTGAAT
GTAATAAGTGATTTGAAAAAAATTGAAGATCTTATTCAATCTATGCATAT
TGATGCTACTTTATATACGGAAAGTGATGTTCACCCCAGTTGCAAAGTAA
CAGCAATGAAGTGCTTTCTCTTGGAGCTCCAGGTAATTAGCTTAGAATCT
GGCGACGCCAGCATCCACGATACAGTTGAGAATCTGATCATTCTCGCAAA
CAACTCTCTGTCTTCTAACGGCAACGTGACAGAGTCTGGCTGTAAGGAGT
GTGAGGAGCTGGAGGAGAAGAACATCAAGGAGTTTCTGCAGTCTTTTGTG
CACATCGTGCAGATGTTTATCAACACCTCTTGA

A further object of the present invention relates to a nucleic acid that encodes for an immunocytokine of the present invention.

Typically, said nucleic acid is a DNA or RNA molecule, which may be included in any suitable vector, such as a plasmid, cosmid, episome, artificial chromosome, phage or a viral vector.

So, a further object of the invention relates to a vector comprising a nucleic acid of the present invention.

Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a host cell which has been transfected, infected or transformed by a nucleic acid and/or a vector according to the invention.

The nucleic acids of the invention may be used to produce an antibody of the present invention in a suitable expression system. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts. Mammalian host cells include Chinese Hamster Ovary (CHO cells) including dhfr-CHO cells (described in Urlaub and Chasin, 1980) used with a DHFR selectable marker, CHOK1 dhfr+ cell lines, NSO myeloma cells, COS cells and SP2 cells, for example GS CHO cell lines together with GS Xceed™ gene expression system (Lonza), or HEK cells.

The present invention also relates to a method of producing a recombinant host cell expressing a polypeptide according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

The host cell as disclosed herein are thus particularly suitable for producing the polypeptide of the present invention. Indeed, when recombinant expression are introduced into mammalian host cells, the polypeptides are produced by culturing the host cells for a period of time sufficient for expression of the polypeptide in the host cells and, optionally, secretion of the polypeptide into the culture medium in which the host cells are grown. The polypeptides can be recovered and purified for example from the culture medium after their secretion using standard protein purification methods.

Therapeutics Uses of the Present Invention:

A further object of the invention relates to the use of the IL-15/IL-15 receptor alpha (IL-15Rα) fusion protein of the present invention as a drug. In particular, said fusion proteins are particularly suitable of the treatment of cancer or infectious diseases. Thus a further object of the present invention relates to a method of therapy in a subject in need thereof comprising administering to the patient a therapeutically effective amount of the IL-15/IL-15 receptor alpha (IL-15Rα) fusion protein of the present invention

A further object of the present invention relates the use of the immunocytokine of the present invention as a drug. Thus a further object of the present invention relates to a method of therapy in a subject in need thereof comprising administering to the patient a therapeutically effective amount of the immunocytokine of the present invention.

In particular, the anti-PD-1 immunocytokines according to the present invention are particularly suitable for enhancing the proliferation, migration, persistence and/or activity of T cells in a subject in need thereof. In particular, the anti-PD-1 immunocytokines according to the present invention are particularly suitable for enhancing the proliferation, migration, persistence and/or activity of T CD4⁺ cells. In particular, the anti-PD-1 immunocytokines according to the present invention are particularly suitable for enhancing the proliferation, migration, persistence and/or activity of T CD8⁺ cells. In particular, the anti-PD-1 immunocytokines according to the present invention are particularly suitable for enhancing the proliferation, migration, persistence and/or activity of Gamma delta T cells. In particular, the anti-PD-1 immunocytokines according to the present invention are particularly suitable for enhancing the proliferation, migration, persistence and/or activity of CAR-T cells.

Thus a further object of the present invention provides a method of therapy in a subject in need thereof, comprising administering to the subject a therapeutically effective amount at least one anti-PD-1 immunocytokine, wherein said administration enhances the proliferation, migration, persistence and/or activity of T cells in the subject.

More particularly, the present invention provides a method of reducing T cell exhaustion in a subject in need thereof comprising administering to the subject a therapeutically effective amount at least one anti-PD-1 immunocytokine of the present invention.

In some embodiments, anti-PD-1 immunocytokines of the present invention are particularly suitable for enhancing the proliferation, migration, persistence and/or activity of T cells specific for an antigen.

In some embodiments, the antigen is a tumor-associated antigen (TAA). Examples of TAAs include, without limitation, melanoma-associated Ags (Melan-A/MART-1, MAGE-1, MAGE-3, TRP-2, melanosomal membrane glycoprotein gp100, gp75 and MUC-1 (mucin-1) associated with melanoma); CEA (carcinoembryonic antigen) which can be associated, e.g., with ovarian, melanoma or colon cancers; folate receptor alpha expressed by ovarian carcinoma; free human chorionic gonadotropin beta (hCGP) subunit expressed by many different tumors, including but not limited to ovarian tumors, testicular tumors and myeloma; HER-2/neu associated with breast cancer; encephalomyelitis antigen HuD associated with small-cell lung cancer; tyrosine hydroxylase associated with neuroblastoma; prostate-specific antigen (PSA) associated with prostate cancer; CA125 associated with ovarian cancer; and the idiotypic determinants of a B-cell lymphoma that can generate tumor-specific immunity (attributed to idiotype-specific humoral immune response), Mesothelin associated with pancreatic, ovarian and lung cancer, P53 associated with ovarian, colorectal, non small cell lung cancer, NY-ESO-1 associated with testis, ovarian cancer, EphA2 associated with breast, prostate, lung cancer, EphA3 associated with colorectal carcinoma, Survivin associated with lung, breast, pancreatic, ovarian cancer, HPV E6 and E7 associated with cervical cancer, EGFR associated with NSCL cancer. Moreover, Ags of human T cell leukemia virus type 1 have been shown to induce specific cytotoxic T cell responses and anti-tumor immunity against the virus-induced human adult T-cell leukemia (ATL). Other leukemia Ags can equally be used. Tumor-associated antigens which can be used in the present invention are disclosed in the book “Categories of Tumor Antigens” (Hassane M. et al Holland-Frei Cancer Medicine (2003). 6th edition.) and the review Gregory T. et al (“Novel cancer antigens for personalized immunotherapies: latest evidence and clinical potential” Ther Adv Med Oncol. 2016; 8(1): 4-31) all of which are herein incorporated by reference. In some embodiments, the tumor-associated antigen is melanoma-associated Ags.

In some embodiments, the antigen is an infectious disease antigen. Thus in some embodiments, the antigen is selected from infectious disease antigens selected from bacterial, viral, parasitic, and fungal antigens. Typically, the antigen is at least one viral antigen. For example, at least one viral antigen comprise peptides from an adenovirus, retrovirus, picornavirus, herpesvirus, rotaviruses, hantaviruses, coronavirus, togavirus, flavirvirus, rhabdovirus, paramyxovirus, orthomyxovirus, bunyavirus, arenavirus, reovirus, papilomavirus, parvovirus, poxvirus, hepadnavirus, rotovirus or spongiform virus. In some embodiments, the at least one viral antigen comprise peptides from at least one of HIV, CMV, hepatitis A, B, and C, influenza; measles, polio, smallpox, rubella, respiratory syncytial, herpes simplex, varicella zoster, Epstein-Barr, Japanese encephalitis, rabies, flu, or cold viruses. In some embodiments, said viral antigens are selected from one or more of the following antigenic domains: HIV-1 Gag p24, Nef, and Gag p17 (including the combination of the three antigens called GNG, see the detailed of the amino acid sequence below) or the combination of HVP16 E6 and HPV16 E7 antigens (HPV 16 E6/E7) (also as referred to HPV, see the detailed amino acid sequence below). In some embodiments, said viral antigens are selected from one or more of the following HIV antigenic domains: Gag p17 (17-35), Gag p17-p24 (253-284) and Nef (116-145), Pol 325-344 (RT 158-188) and Nef (66-97). In some embodiments, said viral antigens are selected from the following combination of the 5 HIV antigenic domains: Gag p17 (17-35), Gag p17-p24 (253-284) and Nef (116-145), Pol 325-344 (RT 158-188) and Nef (66-97).

A further object of the present invention relates to an anti-PD-1 immunocytokine of the present invention for use in the treatment of a cancer. Indeed, the anti-PD-1 immunocytokines of the present invention are particular suitable for enhancing the proliferation, migration, persistence and/or activity of tumor infiltrating cytotoxic T lymphocytes.

In some embodiments, the method of the present invention is suitable for the treatment of a cancer characterized by a high tumor infiltration of cytotoxic T lymphocytes that express PD-1. Typically said tumor-infiltration of cytotoxic T lymphocytes is determined by any conventional method in the art. For example, said determination comprises quantifying the density of cytotoxic T lymphocytes that express PD-1 in a tumor sample obtained from the patient. In some embodiments, the quantification of density of cytotoxic T lymphocytes that express at least one immune checkpoint protein is determined by immunohistochemistry (IHC). For example, the quantification of the density of cytotoxic T lymphocytes is performed by contacting the tissue tumor tissue sample with a binding partner (e.g. an antibody) specific for a cell surface marker of said cells. Typically, the quantification of density of cytotoxic T lymphocytes is performed by contacting the tissue tumor tissue sample with a set of binding partners (e.g. an antibody) specific for CD8 and for PD-1.

A further object of the present invention relates to a method of treating cancer in patient in need thereof comprising administering to the patient a therapeutically effective amount of a population of CAR-T cells in combination with a therapeutically effective amount of an anti-PD-1 immunocytokine of the present invention. The anti-PD-1 immunocytokine of the present invention will thus prevent the exhaustion of the population of CAR-T cells that are specific for a tumor associated antigen.

A further object of the present invention relates to an anti-PD-1 immunocytokine of the present invention for use in the treatment of an infectious disease. More particularly, the anti-PD-1 immunocytokine of the present invention are particularly suitable of the treatment of a viral infection. Examples of viral infections treatable by the present invention include those caused by single or double stranded RNA and DNA viruses, which infect animals, humans and plants, such as retroviruses, poxviruses, immunodeficiency virus (HIV) infection, echovirus infection, parvovirus infection, rubella virus infection, papillomaviruses, congenital rubella infection, Epstein-Barr virus infection, mumps, adenovirus, AIDS, chicken pox, cytomegalovirus, dengue, feline leukemia, fowl plague, hepatitis A, hepatitis B, HSV-1, HSV-2, hog cholera, influenza A, influenza B, Japanese encephalitis, measles, parainfluenza, rabies, respiratory syncytial virus, rotavirus, wart, and yellow fever, adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV).

The anti-PD-1 immunocytokine of the present invention are also particularly suitable for vaccination purposes. In particular, said immunocytokines are particularly suitable for potentiating the immune responses induced by a vaccine composition. Thus, the a further object of the present invention relates to a method for eliciting and/or enhancing B-cell and/or T-cell response against a viral or tumor associated antigen, in a subject in need thereof, comprising administering to said subject in need thereof, an anti-PD-1 immunocytokine of the present invention. In some embodiments, the anti-PD-1 immunocytokine of the present invention is administered sequentially or concomitantly to the vaccine. In some embodiments, the anti-PD-1 immunocytokine of the present invention is mixed with a vaccine composition extemporaneously prior to injection of the vaccine composition to the subject.

Thus, in some embodiments, the anti-PD-1 immunocytokine of the present invention is administered to the subject in combination with at least one antigen for vaccination purposes. Typically, said antigen is a viral antigen. In some embodiments, said viral antigens are selected from one or more of the following antigenic domains: HIV-1 Gag p24, Nef, and Gag p17 (including the combination of the three antigens called GNG, see the detailed of the amino acid sequence below) or the combination of HVP16 E6 and HPV16 E7 antigens (HPV 16 E6/E7) (also as referred to HPV, see the detailed amino acid sequence below). In some embodiments, said viral antigens are selected from one or more of the following HIV antigenic domains: Gag p17 (17-35), Gag p17-p24 (253-284) and Nef (116-145), Pol 325-344 (RT 158-188) and Nef (66-97). In some embodiments, said viral antigens are selected from the following combination of the 5 HIV antigenic domains: Gag p17 (17-35), Gag p17-p24 (253-284) and Nef (116-145), Pol 325-344 (RT 158-188) and Nef (66-97).

In some embodiments, the antigen or the plurality of antigen are conjugated to DC-targeting antibody, in particular anti-CD40 antibodies. Thus, in some embodiments, the anti-PD-1 immunocytokine of the present invention is administered to the subject in combination with at least one antigen conjugated to an anti-CD40 antibody for vaccination purposes. Typically, said anti-CD40 antibody comprises one or more antigens fused or conjugated or coupled by non-covalent coupling to either the corresponding heavy or light chain of said antibody or its antigen-binding fragment. Said antigens may be conjugated directly to a polypeptide chain of the anti-CD40 antibody, for example at the C-terminal end of a polypeptide chain of the anti-CD40 antibody, and, optionally via peptide linker, such as FlexV1, as described above. They can be also coupled by non-covalent coupling, for example as included in cohesin fusion proteins for coupling with dockerin domain.

Additionally, the vaccination methods as disclosed herein may also comprise the administration of one or more adjuvants. The adjuvants may be attached or conjugated directly or indirectly to one or more of the vaccine components, such as an antigen or an anti-CD40 antibody as described above. In some embodiments, the adjuvants may be provided or administered separately from the vaccine composition. In some embodiments the adjuvant is poly ICLC, CpG, LPS, Immunoquid, PLA, GLA or cytokine adjuvants such as IFNα. In some embodiments the adjuvant may be a toll-like receptor agonist (TLR). Examples of TLR agonists that may be used comprise TLR1 agonist, TLR2 agonist, TLR3 agonist, TLR4 agonist, TLR5 agonist, TLR6 agonist, TLR7 agonist, TLR8 agonist or TLR9 agonist.

In some embodiments, the vaccination method of the present invention is used to prevent healthy subject to be infected by said virus, comprising administering a viral vaccine in combination with an anti-PD-1 immunocytokine of the present invention, e.g. to a healthy subject, not infected by said virus (preventive treatment). In some embodiments, the vaccination method of the present invention is used in a method of treating a patient suffering from a viral infection comprising administering to the patient a viral vaccine in combination with an anti-PD-1 immunocytokine of the present invention.

Pharmaceutical Compositions of the Present Invention:

A further object of the present invention relates to a pharmaceutical comprising the IL-15/IL-15 receptor alpha (IL-15Rα) fusion protein of the present invention with a pharmaceutically acceptable carrier.

A further object of the present invention relates to a pharmaceutical comprising the immunocytokine (e.g. the anti-PD-1 immunocytokine) of the present invention with a pharmaceutically acceptable carrier.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g., 1 ml for intramuscular), and between about 1 ng to about 100 mg, e.g., about 50 ng to about 30 mg or more preferably, about 5 mg to about 25 mg, of the polypeptide of the invention.

The disclosure also relates to a vaccine composition comprising the anti-PD-1 immunocytokine of the present invention. In some embodiments, the vaccine composition comprises at least one antigen as above described that can be optionally conjugated to an anti-CD40 antibody as described above. In some embodiments, the vaccine composition of the present invention may further comprise at least one adjuvant. Examples of adjuvants that may be effective include but are not limited to: aluminum hydroxide, N-acetyl-muramyl-L-threonyl-D-isoglutamine (thr-MDP), N-acetyl-nor-muramyl-L-alanyl-D-isoglutamine, MTP-PE and RIBI, which contains three components extracted from bacteria, monophosphoryl lipid A, trehalose dimycolate and cell wall skeleton (MPL+TDM+CWS) in a 2% squalene/Tween 80 emulsion. Other examples of adjuvants include DDA (dimethyldioctadecylammonium bromide), Freund's complete and incomplete adjuvants and QuilA. In addition, immune modulating substances such as lymphokines (e.g., IFN-[gamma], IL-2 and IL-12) or synthetic IFN-[gamma] inducers such as poly I:C or poly ICLC (Hiltonol) can be used in combination with adjuvants described herein. In some embodiments, the adjuvant may be selected among poly ICLC, CpG, LPS, Immunoquid, PLA, GLA or cytokine adjuvants such as IFNα. In some embodiments the adjuvant may be a toll-like receptor agonist (TLR). Examples of TLR agonists that may be used comprise TLR1 agonist, TLR2 agonist, TLR3 agonist, TLR4 agonist, TLR5 agonist, TLR6 agonist, TLR7 agonist, TLR8 agonist or TLR9 agonist. The vaccine preparation may be prepared as injectables, either as liquid solutions or suspensions; solid forms suitable for solution in, or suspension in, liquid prior to infection can also be prepared. The preparation may be emulsified, encapsulated in liposomes. The active immunogenic ingredients are often mixed with carriers which are pharmaceutically acceptable and compatible with the active ingredient.

Administration of vaccines or pharmaceutical compositions according to the present disclosure will be via any common route so long as the target tissue is available via that route in order to maximize the delivery of antigen to a site for maximum (or in some cases minimum) immune response. Administration of vaccines will generally be by orthotopic, intradermal, mucosally, subcutaneous, intramuscular, intraperitoneal or intravenous injection. Other areas for delivery include: oral, nasal, buccal, rectal, vaginal or topical. Vaccines of the disclosure are preferably administered parenterally, by injection, for example, either subcutaneously or intramuscularly.

Vaccines or pharmaceutical compositions of the present disclosure may be administered in a manner compatible with the dosage formulation, and in such amount as will be prophylactically and/or therapeutically effective. The quantity to be administered depends on the subject to be treated, including, e.g., capacity of the subject's immune system to synthesize antibodies, and the degree of protection or treatment desired. Suitable dosage ranges are of the order of several hundred micrograms active ingredient per vaccination with a range from about 0.1 mg to 1000 mg, such as in the range from about 1 mg to 300 mg, or in the range from about 10 mg to 50 mg.

A vaccine may typically be given in a single dose schedule or in a multiple dose schedule. A multiple dose schedule is one in which a primary course of vaccination may include, e.g., 1-10 separate doses, followed by other doses given at subsequent time intervals required to maintain and or reinforce the immune response, for example, at 1-4 months for a second dose, and if needed, a subsequent dose(s) after several months. Periodic boosters at intervals of 1-5 years, usually 3 years, are desirable to maintain the desired levels of protective immunity. The course of the immunization can be followed by in vitro proliferation assays of peripheral blood lymphocytes (PBLs) co-cultured with the antigen, and by measuring the levels of IFN-[gamma] released from the primed lymphocytes. The assays may be performed using conventional labels, such as radionucleotides, enzymes, fluorescent labels and the like. These techniques are known to one skilled in the art and can be found in U.S. Pat. Nos. 3,791,932, 4,174,384 and 3,949,064.

A vaccine may be provided in one or more “unit doses”. Unit dose is defined as containing a predetermined-quantity of the vaccine calculated to produce the desired responses in association with its administration, i.e., the appropriate route and treatment regimen. The quantity to be administered, and the particular route and formulation, are within the skill of those in the clinical arts. The subject to be treated may also be evaluated, in particular, the state of the subject's immune system and the protection desired. A unit dose need not be administered as a single injection but may include continuous infusion over a set period of time. The amount of vaccine delivered can vary from about 0.001 to about 0.05 mg/kg body weight, for example between 0.1 to 5 mg per subject.

The invention will be further illustrated by the following figures and examples. However, these examples and figures should not be interpreted in any way as limiting the scope of the present invention.

### FIGURES

FIG. 1: Anti-PD-1/IL-15/Rα immunocytokine designs that were profiled for functional activity and in vivo pharmacokinetic properties. Immunocytokines consist of (A) a knob-into-hole bi-specific design with one arm targeting PD-1 and the second delivering IL-15 and the IL-15 receptor a sushi domain (IC:K-in-H), (B) anti-PD-1 antibody with a C-terminal dockerin fusion that binds tightly to an IL-15/Rα fusion through an N-terminal cohesin domain (IC:Doc/Coh) and (C) an anti-PD-1 antibody with a C-terminal fusion of IL-15 and IL-15 Rα joined through flexible loops (IC:Fusion).

FIG. 2: Anti-PD-1/IL-15/Rα immunocytokines have enhanced recovery of exhausted CD8⁺ T cell proliferation relative to Keytruda. Recovery of proliferation in HIV-specific CD8⁺ T cells following stimulation with an HIV derived peptide. Results from multiple CFSE experiments are shown with enhance proliferation shown relative to Keytruda that was used as a positive control reference in each study.

FIG. 3: Comparison of IL-15/IL-15Rα immunocytokines directly fused to either a classical blocking (Keytruda) or a non-blocking (135C H1cL1c) anti-PD-1 antibody. Recovery of proliferation in HIV-specific CD8⁺ T cells following stimulation with an HIV derived peptide.

FIG. 4: Antigen-specific CD8 T cells proliferation observed in cells stimulated in the presence of Keytruda or Anti-PD-1/IL-15/Rα immunocytokines. PBMCs from a viremic HIV donor were stimulated with an HIV antigenic peptide and left in culture for 6-days before staining cells with a pentamer MHC-peptide complex.

FIG. 5: Pharmacokinetic profile of the anti-PD-1 antibody Keytruda in comparison with different anti-PD-1/IL-15/Rα immunocytokines. Total human IgG antibody was measured in the Keytruda samples and human IgG antibody with bound IL-15 was monitored for the immunocytokines.

FIG. 6. Experimental design. PBMCs from cART-patients at wk16 post vaccination (samples n=15) were stimulated with Gag-P24 peptides pools in the presence or not of, αPD1, IL-15/IL-15Rα, αPD1+IL-15/IL-15Rα or αPD1 fused to IL-15/IL-15Rα (fusion). After 44 hrs of stimulation cells were collected and stained then analyzed by flow cytometry to depict CD4⁺OX40⁺CD25⁺Foxp3⁺CD39⁺ specific-Tregs and CD4⁺OX40⁺CD25⁺Foxp3⁻CD39 specific effectors. Simultaneously, we assessed CD8⁺Pentamers⁺HIV-1 specific cell proliferation and cytokines production after a 6-day in vitro stimulation.

FIG. 7. CD4⁺HIV-1 specific responses. PBMCs from cART-patients at wk16 post vaccination (n=15) were stimulated with Gag P24 peptides pools in the presence or not of immuno-cytokines as explained in FIG. 2. After 44 hrs of stimulation cells were analyzed by flow cytometry to depict CD4⁺OX40⁺CD25⁺Foxp3⁺CD39⁺ specific-Tregs and CD4⁺OX40⁺CD25⁺Foxp3⁻CD39⁻ specific-effectors.

FIG. 8. CD8⁺ specific responses. PBMCs from Dalia1 samples (n=5, but only 3 are shown) were CF SE-labeled and stimulated with Gag-P24 peptides pools in vitro and cultured in the presence (or not) of either αPD1 alone or α-PD1_IL-15/IL-15Rα fusion. Flow cytometry stainings show gating strategy for CD8⁺Pentamers⁺ specific cells (A), proliferation (B) and cytokines production (C).

FIG. 9. Experimental design for in vitro anti-CD40.HIV5pep-DC vaccine. PBMCs from cART patients (Physioph cohort, Hopital Henri-Mondor Creteil) were used. Samples (n=10) were used for CD14⁺ monocytes isolation (Miltenyi beads), differentiated, matured, activated and loaded with anti-CD40.HIV5pep (DC-vaccine) as performed for the Dalia1 trial (figurel). Experiments using PBMCs stimulated with Gag-P24 peptides pools or co-cultured with anti-CD40.HIV5pep in the presence (or not) of α-PD1 IL-15/IL-15Rα fusion during 44 hrs, have been performed. Gating strategies for CD4⁺OX40⁺CD25⁺ effectors and Tregs as applied above have been used.

FIG. 10. CD4⁺HIV-1 specific responses. Read-out experiments for FIG. 9 (above) showing Teff and Tregs specific cell frequencies. PBMCs stimulated with Gag-P24 peptides pools or co-cultured with anti-CD40.HIV5pep in the presence (or not) of α-PD1 IL-15/IL-15Rα fusion during 44 hrs, have been performed. Gating strategies for CD4⁺OX40⁺CD25⁺ effectors and Tregs as shown in FIG. 5 have been applied.

FIG. 11. CD4⁺ and CD8⁺HIV-1 specific responses. Read-out experiments for FIG. 9 (above) showing cytokines production from effector CD4⁺ and CD8⁺ T cells. 6 hrs before the end of the 44 hrs (OX40 assay), brefeldinA has been added in order to block intracellular cytokines secretion. Staining for IL-2/TNFα/IFNγ has been performed and cells were analyzed by flow cytometry. Blue histograms show increased cytokine production when α-PD1 IL-15/IL-15Rα fusion was added to PBMCs/anti-CD40.HIV5pep-DC cocultures.

FIG. 12. Immunotherapy induced suppression of tumor growth in the PD-1 HuGEMM in vivo Panc02 tumor model. PD-1 huGEMM mice were implanted with Panc02 tumor cells and upon reaching a mean tumor volume of 100 mm³, were treated twice per week with PBS or one of the four immunotherapies indicated. The 10 mice allocated to each group were monitored for tumor volume twice per week with the mean volume show versus days under therapy.

FIG. 13: The NB01/IL-15/IL-15Rα immunocytokine exert a significant suppression in Panc02 tumor growth from Day 7 to 24 following immunotherapy initiation. The anti-tumor effect of the different therapies was evaluated by calculating the area under the curve (AUC) from Day 7 to Day 24 following initiation of the study. Statistical differences between the PBS control and the individual therapies was determined using the non-parametric Mann-Whitney test. Analysis was performed using Graphpad Prism 8.3.0.

FIG. 14: NB01/IL-15/IL-15Rα immunocytokine therapy prolongs the survival of Panc02 implanted PD-1 HuGEMM mice. The percentage of mouse survival over time following initiation of therapy in Panc02 implanted mice was evaluated by Kaplan-Meier survival curve analysis. The log rank test confirmed that both the NB01/IL-15/IL-15Rα immunocytokine monotherapy and the Keytruda+IL-15/IL-15Rα ALT-803 super agonist dual therapy significantly increased the time of mouse survival. In contrast, neither Keytruda monotherapy alone nor the Keytruda/IL-15/IL-15Rα immunocytokine monotherapy significantly prolonged survival of the Panc02 implanted mice. Analysis was performed using Graphpad Prism 8.3.0.

### EXAMPLE 1

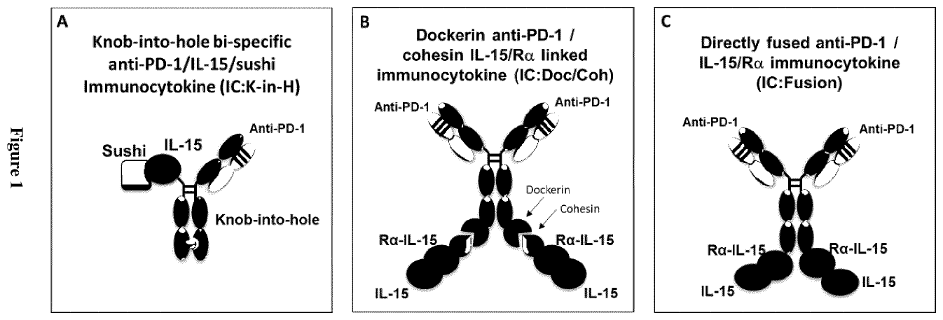

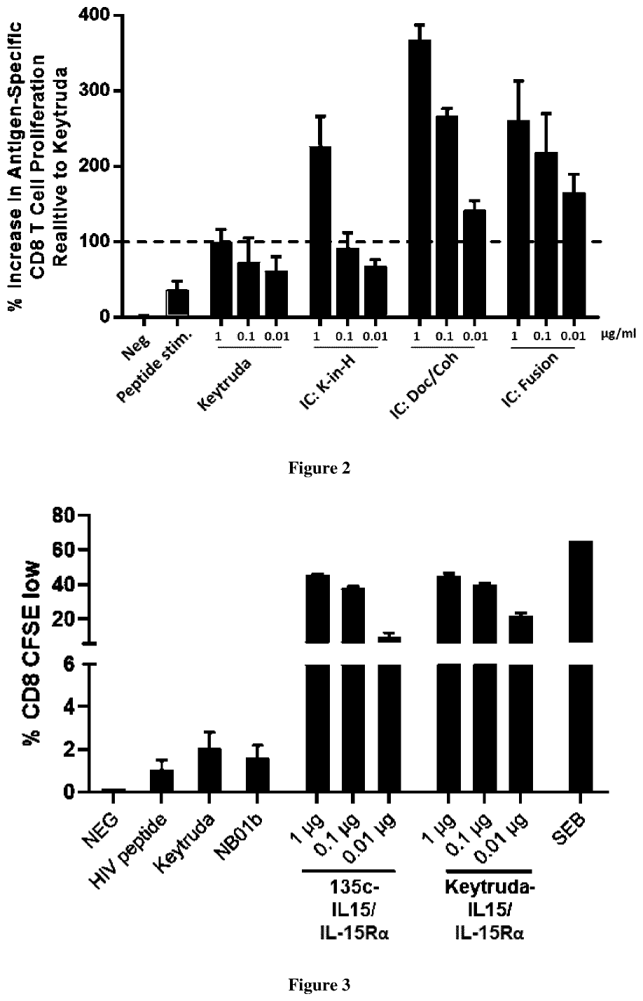

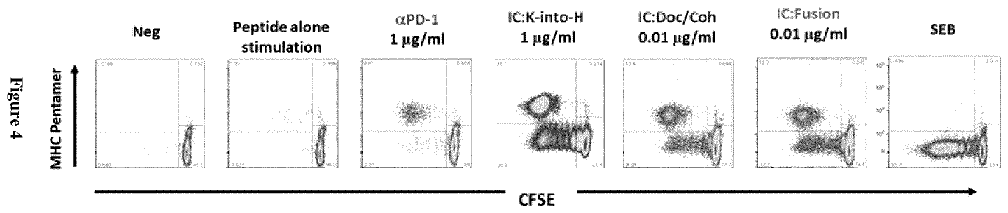

In the design of immunocytokines, our focus was on identifying therapeutic agents that had improved potency in enhancing antigen specific T cell proliferation relative to anti-PD-1 therapy and a sufficiently long in vivo half-life. A second consideration was the production yield and biophysical stability of these immunocytokines that will facilitate the advancement of a promising candidate for in vivo testing and pre-clinical profiling. Three separate immunocytokine designs were evaluated as illustrated in FIG. 1. The immune-enhancing activity of the novel anti-PD-1/IL-15/Rα was evaluated in vitro in a highly standardized CF SE proliferation assay using blood mononuclear cells from chronically infected HIV infected donors. HIV-specific T cells from these donors have been exposed to high levels of antigen over an extended period of time and as such are functionally exhausted, express high levels of the PD-1 immune checkpoint inhibitors and have a poor proliferative response in the presence of antigen-specific stimulation. Stimulation of the blood mononuclear cells with HIV derived peptides followed by six days in culture led to an increase in CFSE low CD8 T cells that have undergone proliferation. Addition of the classical PDL-1 blocking anti-PD-1 Ab Keytruda led to an enhanced level of proliferation relative to the peptide alone control thus indicating that anti-PD-1 Abs recover CD8⁺ T cells from exhaustion (FIG. 2). The three different immunocytokine constructs tested in parallel gave a minimum of a 2 to 4-fold increased level of CD8⁺ T cell proliferation relative to the Keytruda control anti-PD-1 antibody. Importantly, the IC:Doc/Coh and IC:Fusion constructs had significantly improved immune-enhancing effects relative to Keytruda even at 100-fold lower protein concentrations. The IC:K-in-H construct induced increased levels of CD8 T cell proliferation relative to Keytruda at 1 μg/ml, however IC:K-in-H was >10-fold less potent than IC:Doc/Coh and IC:Fusion constructs. In a separate experiment, an additional immunocytokine was generated using the IC:Fusion construct design in combination with the 135C H1cL1c anti-PD-1 antibody that is non-blocking of the PD-1/PDL-1 interaction (Fenwick et al, 2019). The functional activity of 135c directly fused to IL-15/IL-15Rα in the CFSE proliferation assay was found to be comparable to the IC:Fusion that is a Keytruda-IL-15/IL-15Rα fusion (FIG. 3). Interestingly, with the blood mononuclear cells from this viremic HIV infected donors, the two different immunocytokines at 0.1 μg/ml induced up to a 20-fold increase in CD8 T cell proliferation relative to Keytruda at 5 μg/ml.

### EXAMPLE 2

The increased level of CD8⁺ T cell proliferation in the presence of the different immunocytokine highlights their superior functional activity relative to anti-PD-1 therapy alone. However, equally important is that CD8⁺ T cell expansion is specific for the HIV peptide antigen used in the stimulation. The specificity of the enhanced CD8⁺ T cell proliferation in the presence of immunocytokines was determined by staining of CFSE low proliferating CD8⁺ T cells with the pentamer MHC-HIV peptide complex. Flow cytometry results in FIG. 4 show that with Keytruda, IC:K-in-H, IC:Doc/Coh and IC:Fusion, the majority (60-80%) of the proliferating CD8⁺ T cells are MHC pentamer positive and specific for the HIV peptide antigen used in the stimulation. In contrast, stimulations with the SEB T cell superantigen results in only 2% of the proliferating CD8⁺ T cells staining positive for the MHC-HIV peptide complex. These pentamer staining studies confirm that the immunocytokine complexes induce a strong and specific induction CD8⁺ T cells proliferation in the presence of antigenic stimulation.

### EXAMPLE 3

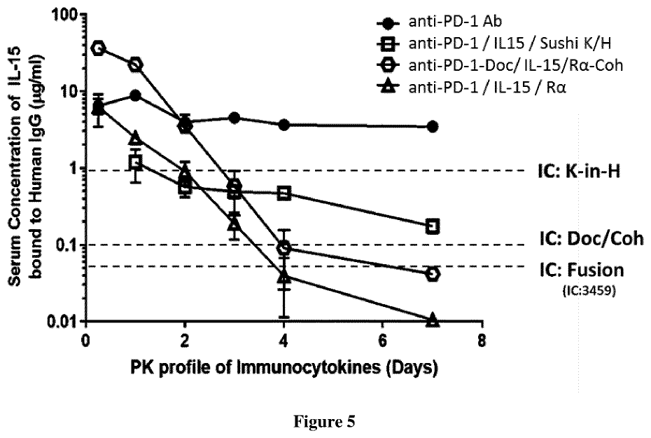

To further characterize these immunocytokines as therapeutic agents, pharmacokinetic studies were performed in C57BL/6 mice that were dosed with the 2 mg/kg of the different drugs and serum samples were collected over the following 7 days. PK properties of Keytruda and the three immunocytokine were determined using Luminex assays to detect human IgG or human IgG with bound IL-15 over the course of the study (FIG. 5). The IC:K-in-H construct, with a more native antibody structure relative to the other immunocytokines, had an in vivo half-life that was closest to the Keytruda antibody (tin, —5 days). However, the drug exposure was also 1 log lower than Keytruda with equivalent doses administered to the mice. Given that the IC:K-in-H construct is only about 10-fold more potent than Keytruda, the inferior PK is likely to negate any functional advantages offered by this immunocytokine. The IC:Doc/Coh and IC:Fusion immunocytokines have a terminal half-lives of approximately 1 day and are more rapidly cleared from mice compared to Keytruda. However, this half-life is significantly longer that the <1 hr half-life reported for IL-15 in vivo and drug levels of these two immunocytokines at day 7 are still be high (>0.01 μg/ml) relative to the significantly improved in vitro functional effect of IC:Doc/Coh and IC:Fusion compared to Keytruda. In contrasting the different properties of the three immunocytokines evaluated, the IC:Fusion has the best overall profile with: 1) a consistent >2-fold improved functional activity relative to Keytruda over a 2 log range in concentration, 2) antigen specific enhanced proliferation of CD8⁺ T cells as demonstrated by MHC petamer staining, and 3) PK profile that would support a once weekly dosing regimen. An additional advantage of the IC:Fusion construct compared to IC:Doc/Coh, is that it lacks the dockerin and cohesin domains that may be prone to increased immunogenic and would be detrimental to the PK profile in animals that were treated with repeat dosing.

### EXAMPLE 4

Based on our previous results, we reasoned that therapeutic DC-based vaccination could be combined with an immuno-modulatory cytokine fused to an immune checkpoint inhibitor in order to increase T cell responses. To this end, we used an αPD-1 monoclonal antibody (Keytruda, a clinical molecule) that proved its' potency in the cancer field and IL-15/IL-15Rα, a cytokine known to impact effector CD8⁺ T cell proliferation.

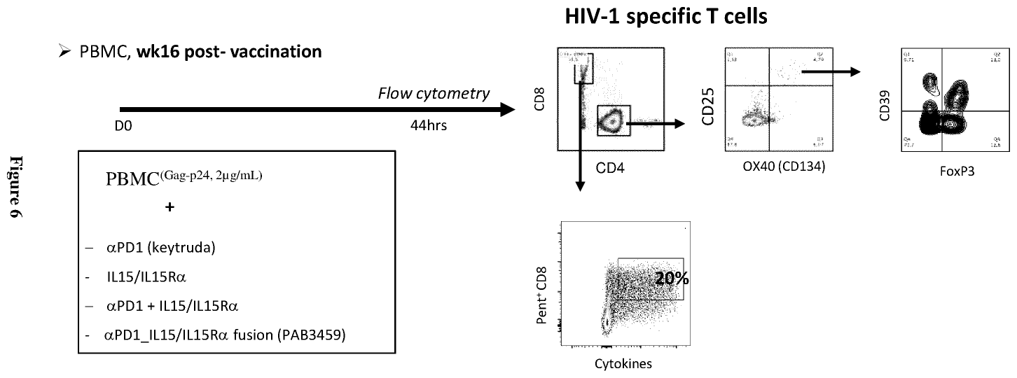

PBMCs from weekly post-vaccination (Dalia1, n=15) were stimulated in vitro with Gag-P24 peptides pools in the presence or not of αPD-1_IL-15/IL-15Rα fusion, αPD1 alone, IL-15/IL-15α alone or αPD-1+IL-15/IL-15α (refer to experimental design, FIG. 6). After 44 hrs stimulation, CD4⁺OX40⁺CD25⁺HIV-1 specific cells have been analyzed.

We demonstrate that in the presence of α-PD-1_IL-15/IL-15Rα fusion, Gag-P24 stimulated PBMCs from cART HIV-1⁺ vaccinated patients led to a significant increase in HIV-1 specific T cell responses as compared to the other conditions. In FIG. 7, panel A, we show that CD4⁺OX40⁺CD25⁺HIV-1 specific cell frequency, that includes both effectors and regulatory cells (Teff and Tregs), increased significantly when α-PD-1_IL-15/IL-15Rα fusion was added in Gag P24- stimulated PBMCs. Addition of αPD-1 did not increase the frequency CD4⁺ specific cells as compared to Gag P24 stimulated cells alone, while IL-15/IL-15Rα or IL-15/IL-15Rα+αPD-1 addition was slightly better but did not reach the potency obtained with α-PD-1_IL-15/IL-15Rα fusion. In FIG. 7 panel B, we show similar trends when zooming on effectors (OX40⁺CD25⁺CD39⁻Foxp3⁻), and interestingly we observed a significant decrease in Tregs (OX40⁺CD25⁺CD39⁺Foxp3⁺) when α-PD-1_IL-15/IL-15Rα fusion was added. These data demonstrate that fusion of αPD-1 with IL-15/IL-15Rα is a good way to boost HIV-1 specific effector CD4⁺ T cells in vitro.

### EXAMPLE 5

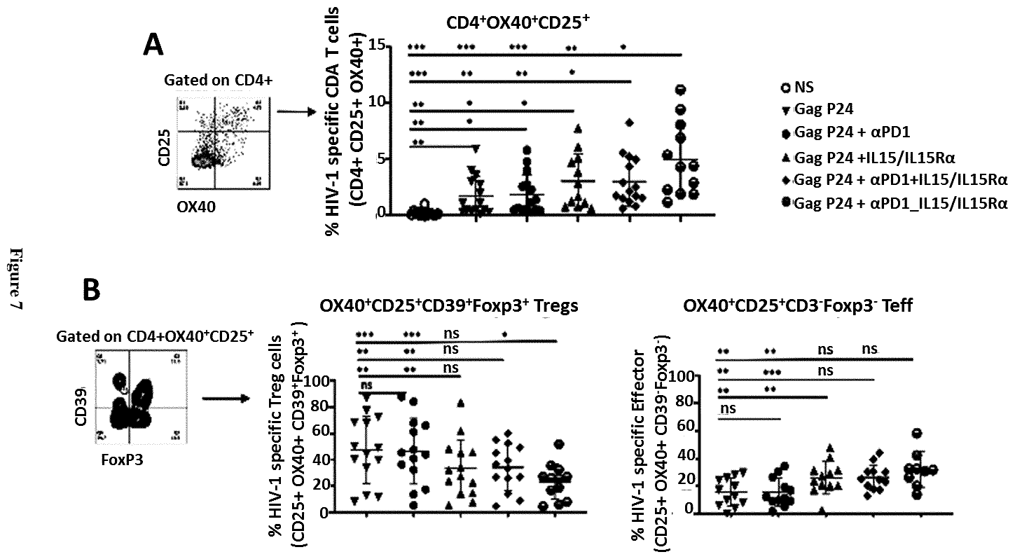

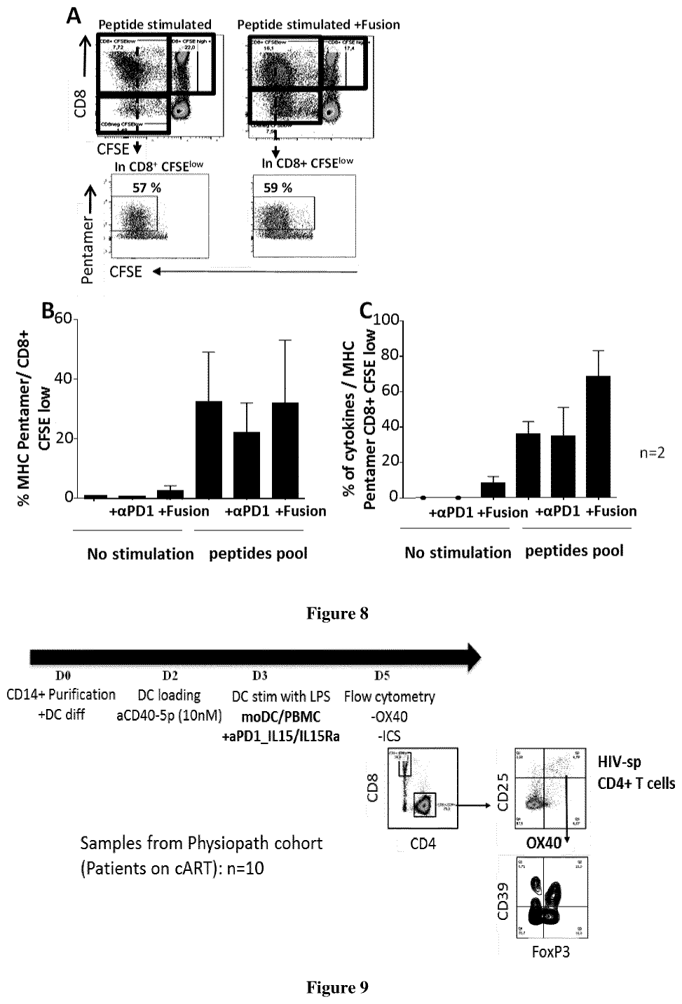

In order to depict CD8⁺HIV-1 specific cells we used pentamers staining (Proimmune, UK) and flow cytometry analyses after in vitro proliferation of PBMCs from 6 patients (Dalia1, week16) that were stimulated with HLA-restricted peptides pools. After a 6 day-in vitro proliferation we performed an overnight re-stimulation with the specific peptides and performed CFSE stainings and flow cytometry to measure CD8⁺ proliferation and intracellular cytokine production (IL-2, TNFα, IFN-γ). The results demonstrated that PBMCs stimulation in the presence of α-PD-1_IL-15/IL-15Rα fusion significantly increased CD8⁺ pentamers⁺ cell proliferation and cytokines production, as compared to αPD1 alone, which showed similar responses when compared to the condition with only peptides stimulation (FIG. 8). The graphs show the results obtained with 3 patients out of 5.

### EXAMPLE 6

In order to demonstrate that αPD-1_IL-15/IL-15Rα fusion could be also used with another DC-based vaccine (anti-CD40.HIV5pep-DC), we performed in vitro experiments where anti-CD40.HIV5pep construct has been loaded on matured and differentiated CD14⁺ monocytes as shown in the experimental design below (FIG. 9 and Cobb et al. JIM 2011), before co-cultures with autologous PBMCs. We used the OX40 assay to depict CD4⁺ specific T cells by flow cytometry as shown in the lower panel of FIG. 9.

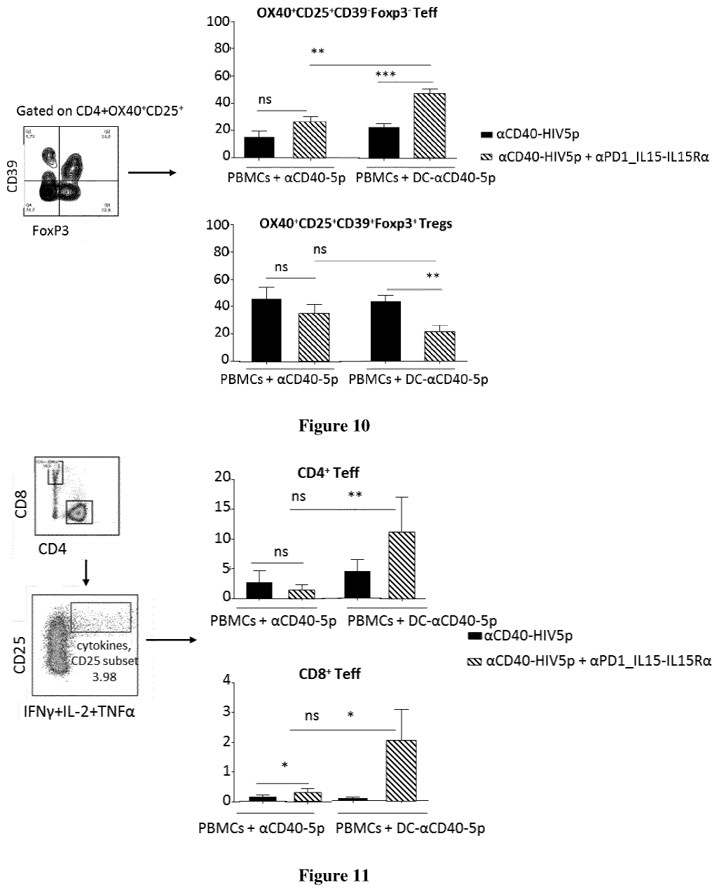

Of note, for the read-out experiments using the OX40 assay, we performed 2 conditions: in the first one we stimulated PBMCs from patients with Gag P24 in the presence or not of αPD-1_IL-15/IL-15Rα fusion and in the second condition we cocultured PBMCs with anti-CD40.HIV5pep-DC in the presence or not of αPD-1_IL-15/IL-15Rα fusion. The results shown in FIG. 10, demonstrated that addition of αPD-1_IL-15/IL-15Rα fusion in both conditions enhanced CD4⁺HIV-1 specific effector cells (blue histograms), but this increase was more significant when PBMCs were stimulated/co-cultured with anti-CD40.HIV5pep-DC. Moreover, we observed a significant decrease in CD4⁺HIV-1 specific Tregs (histogram, lower panel).

Finally, we measured intracellular cytokines (IL-2/TNFα/IFNγ) in both CD4+ and CD8⁺ cells (addition of brefeldin A 6 hrs before the end of the 44 hrs of OX40assay). FIG. 11 shows a significant increase in cytokines production when PBMCs were co-cultured with anti-CD40.HIV5pep_DCs in the presence of αPD-1_IL-15/IL-15Rα fusion.

Altogether these results demonstrate that i) DC-targeting (PBMCs co-cultured with anti-CD40.HIV5pep-DCs) leads to better T cell responses as compared to Gag P24-stimulated PBMCs and ii) combination of αPD-1_IL-15/IL-15Rα fusion boosted CD8⁺-specific responses (proliferation and cytokines production) and decreased CD4⁺-specific Tregs, suggesting that αPD-1_IL-15/IL-15Rα fusion is good tool that can be pushed to the clinic.

### EXAMPLE 7: Efficacy of Anti-PD-1/IL-15/IL15Rα Immunocytokines in the in Vivo Panc02 Mouse Tumor Model

The objective of this study was to evaluate the in vivo therapeutic efficacy of the anti-PD-1/IL-15/IL-15Rα immunocytokine test agents described in this application in the treatment of a subcutaneous Panc02 murine pancreatic cancer xenograft in female HuGEMM hPD-1 mice. The Panc02 tumor cells are poorly immunogenic and represent a challenging tumor model for most cancer immunotherapies.

The HuGEMM PD-1 model performed by CrownBio was developed by knocking-in human exon 2 to replace its mouse PD-1 counterpart. This allows for the in vivo efficacy evaluation of human therapeutic antibodies, which recognize the humanized PD-1 receptor. Mice of age 6-8 weeks were inoculated with 3×10⁶ Panc02 tumor cells in 0.1 mL of PBS and the study was initiated 7 days later when the mean tumor size reaches approximately 100 (70-130) mm³. All animals were randomly allocated to five study group arms based on “Matched distribution” method (StudyDirector™ software, version 3.1.399.19) with 10 mice per group. The five investigational arms of the study included: 1) PBS untreated control, 2) pembrolizumab (Keytruda®) anti-PD-1 twice weekly treatment at 5 mg/kg, 3) pembrolizumab (Keytruda®) at 5 mg/kg+0.1 mg/kg of the IL-15/IL-15Rα ALT-803 super agonist, both administered twice weekly, 4) Keytruda fused to IL-15/IL-15Rα immunocytokine (IC) administered at 2 mg/kg twice weekly and 5) NB01b anti-PD-1 antibody fused to IL-15/IL-15Rα immunocytokine (IC) administered twice weekly at 2 mg/kg. Keytruda used in the study was a clinical lot of antibody purchased from the Lausanne University Hospital. The IL-15/IL-15Rα ALT-803 super agonist and immunocytokines were recombinant proteins produced independently through transient transfection of CHO express or HEK 293T mammalian cell lines. The proteins were expressed with signal sequences that was cleaved upon secretion from the transfected cells. Each of the therapeutic proteins was then purified from the cell medium using a protein A affinity column. Following buffer exchange through dialysis against PBS, therapeutic agents were verified with an limulus amebocyte lysate (LAL) kit from Charles River and endotoxin levels determined to be less than 1 EU/ml. All therapeutic agent were administered intraperitoneal as a solution in PBS buffer. Tumor volumes in all mice was measured twice per week in two dimensions using a caliper, and the volume expressed in mm³ using the formula: volume=(length×width×width)/2. A tumor volume cutoff of 1500 mm³ was selected for establishing mouse survival criteria in this study.

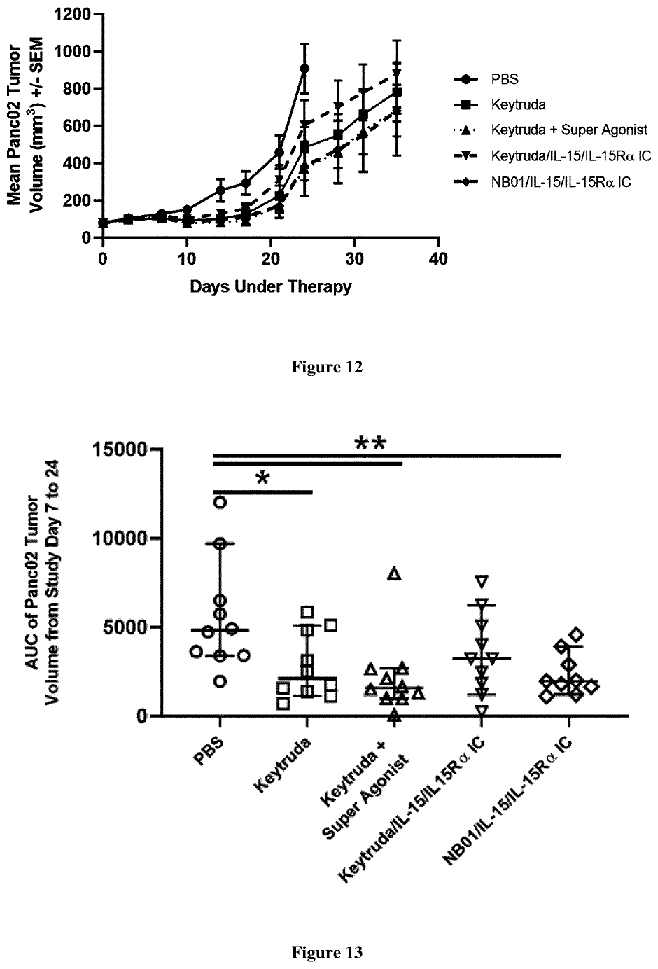

Longitudinal evaluation of mouse tumor volumes in each of the study arms showed that at Day 10 under therapy, all anti-PD-1 based therapies showed signs of tumor suppression relative to the PBS untreated control mice (FIG. 12). The Keytruda alone and Keytruda/IL-15/IL-15Rα IC therapies exerted only transient suppression of tumor growth with increases in mean volume observed at Day 14 of the study. In contrast, Keytruda+ALT-803 super agonist and the NB01/IL-15/IL-15Rα IC therapies demonstrated a prolonged in vivo functional activity with an increase in mean tumor volume observed only seven days later on Day 21 of the study. After this point, the Panc02 cells escaped suppression by the different immunotherapies and tumor growth appeared to progress at a similar rates in all anti-PD-1 based therapeutic arms.

The relative levels of tumor suppression between the different therapies and the PBS arm was evaluated by comparing the tumor volume area under the curve values for Days 7 to 24 of the study (FIG. 13). The strongest tumor suppression over this period was observed in the Keytruda+super agonist and NB01/IL-15/IL-15Rα IC study arms. Relative to the PBS control mice, we observed a significant tumor suppression in Keytruda+super agonist (p=0.0021) and NB01/IL-15/IL-15Rα IC (p=0.0041) therapeutic arms with a less significant reduction in tumor volume for the Keytruda alone therapy (p=0.018).

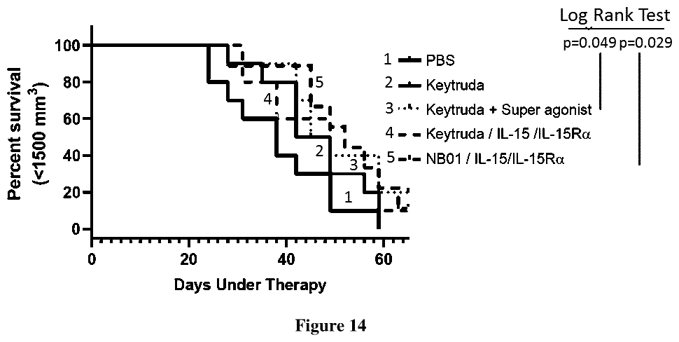

The anti-tumor efficacy of the different therapies was also evaluate using a Kaplan-Meier Survival Curve analysis (FIG. 14). The NB01/IL-15/IL-15Rα immunocytokine monotherapy exerted a statistically significant 14 day increase in mouse survival relative to the PBS control (Log rank test; p=0.029) that was slightly longer than the 12 day increased survival in the dual combination therapy of Keytruda+IL15/IL15Rα super agonist dual therapy (Log rank test; p=0.049). In contrast, both Keytruda and Keytruda/IL-15/IL-15Rα immunocytokine monotherapies did not significantly increase the survival time of mice implanted with the poorly immunogenic Panc02 tumor cells.

Overall, these studies show that the immunocytokine fusion of the NB01b anti-PD-1 antibody with the IL-15 and IL-15Rα has a significant functional activity in both suppressing Panc02 tumor grown and in prolonging mouse survival in comparison to the untreated mice. This demonstrated activity of the immunocytokine was equivalent in efficacy as compared to the Keytruda+super agonist dual therapy.

### REFERENCES

Throughout this application, various references describe the state of the art to which this invention pertains. The disclosures of these references are hereby incorporated by reference into the present disclosure.

1. An IL-15/IL-15 receptor alpha (IL-15Rα) fusion protein comprising i) a IL15-R alpha sushi-containing polypeptide comprising an amino acid sequence having at least 80% of identity with the amino acid sequence of SEQ ID NO:1 ii) a linker having an amino acid sequence as set forth in SEQ ID NO:2 and iii) an IL-15 polypeptide comprising the amino acid sequence having at least at least 80% of identity with the amino acid sequence of SEQ ID NO:3; a nucleic acid encoding the IL-15/IL-15 receptor alpha fusion protein, a vector that comprises the nucleic acid or a host cell which has been transfected, infected or transformed by the nucleic acid.

2. The IL-15/IL-15 receptor alpha fusion protein of claim 1, wherein the IL-15/IL-15 receptor alpha fusion protein has the amino acid sequence as set forth in SEQ ID NO:4.

3. A heavy chain of an antibody that is fused to the IL-15/IL-15 receptor alpha fusion protein of claim 1, a nucleic acid encoding the heavy chain, a vector that comprises the nucleic acid or a host cell which has been transfected, infected or transformed by the nucleic acid.

4. The heavy chain of claim 3, wherein the heavy chain is fused to the IL-15/IL-15 receptor alpha fusion protein via a linker.

5. The heavy chain of claim 4 wherein the linker comprises the amino acid sequence as set forth in SEQ ID NO:5.

6. The heavy chain of claim 5 wherein the linker has the amino acid sequence as set forth in SEQ ID NO:6.

7. The heavy chain of claim 3, wherein the heavy chain is from an antibody having specificity for PD-1.

8. The heavy chain of claim 7, wherein the heavy chain comprises a VH domain as set forth in SEQ ID NO:7, 8 or 9.

9. The heavy chain of claim 7, wherein the heavy chain comprises an IgG Fc region of an IgG4 immunoglobulin.

10. The heavy chain of claim 7, wherein the heavy chain comprises an amino acid sequence as set forth in SEQ ID NO:10, 11 or 12.

11. The heavy chain of claim 7, wherein the heavy chain has the amino acid sequence as set forth in SEQ ID NO:13, 14, or 15.

12. An immunocytokine a heavy chain of an antibody that is fused to the IL-15/IL-15 receptor alpha fusion protein of claim 1, a nucleic acid that encodes the immunocytokine, a vector that comprises the nucleic acid or a host cell which has been transfected, infected or transformed by the nucleic acid.

13. The immunocytokine claim 12, wherein the immunocytokine has specificity for PD-1.

14. The immunocytokine of claim 12 wherein the heavy chain has the amino acid sequence as set forth in SEQ ID NO:13, 14, or 15.

15. The immunocytokine of claim 12, wherein the immunocytokine comprises: a heavy chain a set forth in SEQ ID NO:13 and a light chain a set forth in SEQ ID NO:16, a heavy chain a set forth in SEQ ID NO:14 and a light chain a set forth in SEQ ID NO:17 or a heavy chain a set forth in SEQ ID NO:15 and a light chain a set forth in SEQ ID NO:18.

16. (canceled)

17. (canceled)

18. (canceled)

19. (canceled)

20. The nucleic acid of claim 3, wherein the nucleic acid comprises the nucleic acid sequence as set forth in SEQ ID NO:19 or 20.

21. (canceled)

22. (canceled)

23. (canceled)

24. (canceled)

25. (canceled)

26. (canceled)

27. A method of reducing T cell exhaustion, treating cancer or treating an infectious disease in a subject in need thereof comprising administering to the subject a therapeutically effective amount of at least one anti-PD-1 immunocytokine of claim 13.

28. (canceled)

29. (canceled)

30. The method of claim 27, wherein the infectious disease is a viral infection caused by a single or double stranded RNA or a DNA virus, which infects animals, humans and plants, wherein the single or double stranded RNA or the DNA virus is selected from the group consisting of retroviruses, poxviruses, immunodeficiency virus (HIV), echovirus, parvovirus, rubella virus, papillomaviruses, congenital rubella, Epstein-Barr virus, mumps, adenovirus, AIDS, chicken pox, cytomegalovirus, dengue, feline leukemia, fowl plague, hepatitis A, hepatitis B, HSV-1, HSV-2, hog cholera, influenza A, influenza B, Japanese encephalitis, measles, parainfluenza, rabies, respiratory syncytial virus, rotavirus, wart, yellow fever, adenovirus, a herpesvirus, a poxvirus, a picornavirus, an orthomyxovirus, a paramyxovirus, a coronavirus, a papovavirus, a hepadnavirus, a flavivirus, or a retrovirus.

31. A method for eliciting and/or enhancing B-cell and/or T-cell response against an antigen or a plurality of antigens, in a subject in need thereof, comprising administering to said subject a therapeutically effective amount of the anti-PD-1 immunocytokine of claim 13 in combination with the antigen or the plurality of antigens.

32. The method of claim 31 wherein the antigen or the plurality of antigen are conjugated to a DC-targeting antibody.

33. A pharmaceutical comprising the IL-15/IL-15 receptor alpha (IL-15Rα) fusion protein of claim 1 and a pharmaceutically acceptable carrier.

34. A pharmaceutical comprising the immunocytokine of claim 12 and a pharmaceutically acceptable carrier.

35. A vaccine composition comprising the immunocytokine of claim 13.
